(19) United States
(12) Patent Application Publication
(10) Pub. No.: US 2014/0234342 A1
(43) Pub. Date: Aug. 21, 2014

(54) NKP46-MEDIATED NK CELL TUNING

(71) Applicants: Emilie Narni-Mancinelli, Marseille (FR); Sophie Ugolini, Cassis (FR); Eric Vivier, Cassis (FR)

(72) Inventors: Emilie Narni-Mancinelli, Marseille (FR); Sophie Ugolini, Cassis (FR); Eric Vivier, Cassis (FR)

(73) Assignees: INNATE PHARMA, MARSEILLE (FR); INSERM (INSTITUT NATIONAL DE LA SANTE ET DE LA RECHERCHE MEDICALE), Paris Cedex 13 (FR); UNIVERSITE DE LA MEDITERRANEE AIX-MARSEILLE II, MARSEILLE CEDEX 07 (FR); CENTRE NATIONAL DE LA RECHERCHE SCIENTIFIQUE, PARIS CEDEX 16 (FR)

(21) Appl. No.: 14/127,624

(22) PCT Filed: Jun. 21, 2012
(86) PCT No.: PCT/EP2012/061967

§ 371 Date: May 2, 2014

Related U.S. Application Data
(60) Provisional application No. 61/499,485, filed on Jun. 21, 2011.
(60) Provisional application No. 61/555,579, filed on Nov. 4, 2011.

(51) Int. Cl. C07K 16/18

(52) U.S. Cl. CPC C07K 16/18
USPC 424/172.1; 530/389.8

(57) ABSTRACT

The present invention relates to compounds (e.g. antibodies) that specifically bind and inhibit NKp46. Such compounds are capable, when administered to a mammal, capable of increasing the frequency of reactive and/or active NK cells. The invention also relates to pharmaceutical composition and methods of using the antibodies and compositions to treat or prevent diseases, e.g. cancer, infection, autoimmune diseases, inflammatory diseases and the like.

### FIELD OF THE INVENTION

The present invention relates to compounds (e.g. antibodies) that inhibit NKp46. The invention also relates to cells producing such compounds; methods of making such compounds, and antibodies, fragments, variants, and derivatives thereof; pharmaceutical compositions comprising the same; methods of using the compounds to diagnose, treat or prevent diseases, e.g. cancer, infectious disease, autoimmune diseases, inflammatory diseases and the like.

### BACKGROUND

Natural killer (NK) cells are a subpopulation of lymphocytes that are involved in non-conventional immunity. NK cells provide an efficient immunosurveillance mechanism by which undesired cells such as tumor or virally-infected cells can be eliminated. Characteristics and biological properties of NK cells include the expression of surface antigens including CD16, CD56 and/or CD57, the absence of the alpha/beta or gamma/delta TCR complex on the cell surface; the ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

NK cell activity is regulated by a complex mechanism that involves both activating and inhibitory signals. Several distinct NK-specific receptors have been identified that play an important role in the NK cell mediated recognition and killing of HLA Class I deficient target cells. Natural Cytotoxicity Receptors (NCR) refers to a class of activating receptor proteins, and the genes expressing them, that are specifically expressed in NK cells. Examples of NCRs include NKp30, NKp44, and NKp46 (see, e.g., Lanier (2001) Nat Immunol 2:23-27, Pende et al. (1999) J Exp Med. 190:1505-1516, Cantoni et al. (1999) J Exp Med. 189:787-796, Sivori et al (1997) J. Exp. Med. 186:1129-1136, Pessino et al. (1998) J Exp Med. 188(5):953-60; Mandelboim et al. (2001) Nature 409:1055-1060, the entire disclosures of which are herein incorporated by reference). These receptors are members of the Ig superfamily, and their cross-linking, induced by specific mAbs, leads to a strong NK cell activation resulting in increased intracellular Ca⁺⁺ levels, triggering of cytotoxicity, and lymphokine release, and an activation of NK cytotoxicity against many types of target cells. These findings provide evidence for a central role of these receptors in natural cytotoxicity.

NK cells are negatively regulated by major histocompatibility complex (MHC) class I-specific inhibitory receptors (Karre et al. (1986) Nature 319:675-8; Ohlen et al, (1989) Science 246:666-8). These specific receptors bind to polymorphic determinants of major histocompatibility complex (MHC) class I molecules or HLA and inhibit natural killer (NK) cell lysis. In humans, certain members of a family of receptors termed killer Ig-like receptors (KIRs) recognize groups of HLA class I alleles.

Modulation of NK cell activity has emerged as a promising therapeutic approach. Therapeutic approaches to modulating NK cell activity have included antibodies directed to inhibitory receptors on NK cells to increase NK cell activity by removing KIR-mediated inhibition of the NK cells (see, e.g. WO 2005/003172, WO2006/003179). Depleting antibodies to activating receptors have generally been proposed as means to remove unwanted NK cells, generally in certain inflammatory situations (see, e.g. WO 2005/105848 and EP 1 301 605). Antibodies that act as agonists to activate NCRs have been proposed as a means to augment ADCC in the treatment of cancer and other diseases (see WO2005/009465). However, activating receptors have not been extensively exploited as targets for pharmaceutical modulation. There is therefore a need to provide improved methods of modulating the immune system to treat disease, particularly new approaches of modulating NK cell activity.

### SUMMARY OF THE INVENTION

NK cells are cytotoxic lymphocytes involved in early anti-viral and anti-tumoral immune responses. Using N-ethyl-N-nitrosourea (ENU) mutagenesis in mice, the inventors identified a mutant with hyper-responsive NK cells responsible for an increased resistance to mouse cytomegalovirus infection. Whole genome sequencing revealed a loss-of-function mutation in the Ncr1 gene that encodes the activating NKp46 receptor. Upregulation of activity of NK cells deprived of NKp46 function during their development was demonstrated by genetic complementation in vivo. This upregulation of activity was also mimicked in wild-type animals by blocking NKp46 in vivo with an antibody (saturating NKp46 on NK cells for 11 days) during NK cell development. The inventors further showed that the calibration of NK cell activity via NKp46 engagement was associated with the silencing of the Helios transcription factor. The down-modulation of NK cell responsiveness through NKp46 was key for the subsequent development of anti-viral T cell responses. The results disclosed herein reveal a pivotal role for NKp46 in the tuning of NK cell activity and that NKp46 blockade can be harnessed to enhance NK cell-mediated activity.

The results suggest that rather than seeking to stimulate NKp46 in cancer or to eliminate (deplete) NKp46-positive NK cells in inflammation and autoimmunity, it can be beneficial to augment NK cell activity by blocking NKp46. Blocking NKp46 during NK cell maturation, particularly over a period of time sufficiently long to allow NK cell reprogramming or education, can enhance the frequency of reactive (e.g. toward target cells) and/or active NK cells, and thus enhance NK cells' ability to eliminate tumor cells, infected cells or T cells (e.g. pro-inflammatory T cells).

This invention thus provides a method for treating an individual, the method comprising, consisting essentially of or consisting of: administering to an individual a therapeutically active amount of a compound that inhibits a NKp46 polypeptide. Preferably the compound is a non-depleting antibody (an antibody that does not deplete cells to which it binds). Preferably the antibody is a chimeric, humanized or human antibody. Preferably the antibody comprises a heavy chain constant region of IgG4 isotype. Preferably the compound is administered to an individual for a sufficient period of time to inhibit NKp46 in developing NK cell and cause an increase in the frequency of activated, reactive, cytotoxic and/or IFNγ-producing NK cells in an individual.

Optionally the individual is human having or susceptible to a cancer, infection, undergoing transplantation, or having an inflammatory or autoimmune disorder.

In one embodiment of the methods and uses according to the present invention, the cancer is a solid tumor or a carcinoma. Preferably, the solid tumor is selected from breast cancer, colon cancer, lung cancer, prostate cancer, renal cancer, metastatic or invasive malignant melanoma, brain tumor, ladder cancer and liver cancer. Carcinoma includes bladder, breast, colon, kidney, liver, lung, ovary, pancreas, stomach, cervix, thyroid or skin carcinoma, including squamous cell carcinoma. In one preferred embodiment, the solid tumor is a breast cancer. However, the present invention also contemplates hematopoïetic tumors such as leukemia, acute lymphocytic leukemia, acute lymphoblastic leukemia, B-cell lymphoma, T-cell lymphoma, Hodgkins lymphoma, non-Hodgkins lymphoma, hairy cell lymphoma, Burketts lymphoma, acute and chronic myelogenous leukemias and promyelocytic leukemia. The present invention is also relevant for the treatment of metastasis.

In one embodiment, the inflammatory or autoimmune disorder is a T cell mediated inflammatory or autoimmune disorder, e.g., a disorder involving pro-inflammatory, activated and/or proliferating T cells (e.g. in circulation or in a diseased or inflamed tissue), infiltrating T cells, CD4+ T cells, CD8+ T cells and/or T cells bound by NKp46 (e.g., bound by a soluble NKp46; expressing a ligand bound by NKp46). Examples of inflammatory or autoimmune disorder include systemic lupus erythematosus, Wegener's granulomatosis, autoimmune hepatitis, Crohn's disease, scleroderma, ulcerative colitis, Sjögren's syndrome, Type 1 diabetes mellitus, uveitis, myocarditis, rheumatic fever, ankylosing spondylitis, rheumatoid arthritis, multiple sclerosis, and psoriasis.

The invention also provides a method for eliminating a cell in vivo, e.g., cancer cells, infected cells, pro-inflammatory cells, activated and/or proliferating T cells, the method comprising bringing NK cells that express a NKp46 polypeptide into contact with a compound that inhibits a NKp46 polypeptide, in the presence of said cells to be eliminated. Said bringing into contact preferably comprises administering the compound that inhibits a NKp46 polypeptide to a mammal.

The invention also provides a method for activating an NK cell in vivo, or a method of modulating NK cell maturation (or increasing NK cell reactivity or activity during NK cell maturation) in vivo in a mammal, a method of the method comprising bringing NK cells that express a NKp46 polypeptide into contact with a compound that inhibits a NKp46 polypeptide. Said bringing into contact preferably comprises administering the compound that inhibits a NKp46 polypeptide to the mammal. Activating an NK cell optionally comprises increasing the reactivity or cytoxicity of NK cells toward target cells (infected cells, tumor cells, pro-inflammatory cells, etc.), increasing activation, activation markers (e.g. CD107 expression) and/or IFNγ production in an NK cell, and/or increasing the frequency in vivo of such activated, reactive, cytotoxic and/or activated NK cells.

In another embodiment, the invention provides a method comprising:
(a) determining whether an individual has a cancer, infectious disease, inflammatory or autoimmune disorder; and
(b) if the individual has a cancer, infectious disease, inflammatory or autoimmune disorder, treating the individual with a therapeutically active amount of a compound that inhibits a NKp46 polypeptide.

In another embodiment, the methods comprise:
(a) determining whether an individual has an inflammatory or autoimmune disorder mediated at least in part by T cells, e.g., pro-inflammatory, activated and/or proliferating T cells (e.g. in circulation or in a diseased or inflamed tissue), infiltrating T cells; and
(b) if the individual has an inflammatory or autoimmune disorder mediated at least in part by said T cells, treating the individual with a therapeutically active amount of a compound that inhibits a NKp46 polypeptide.

The invention also provides a non-depleting anti-NKp46 antigen-binding compound that binds and inhibits the function of a human NKp46 polypeptide. This invention provides novel and useful antigen-binding compounds that specifically bind to NKp46 and when administered to a mammal lead to an increased frequency of reactive and/or active NK-cells in vivo. Preferably the antigen-binding compound inhibits NKp46 signaling in an NK cell, e.g, the antigen-binding compound inhibits ligand-induced NKp46 signaling. Optionally the antigen-binding compound inhibits binding of a natural ligand of NK46 (e.g. a soluble or immobilized ligand or a ligand expressed on a cell) to an NKp46 polypeptide. Optionally the antigen-binding compound inhibits NKp46-mediated silencing of helios transcription factor in an NK cell (e.g. the antigen-binding compound leads to an increase of helios expression or activity in a developing NKp46-expressing NK cell, compared to the level observed in the absence of antigen-binding compound). In one embodiment, the antibodies have binding affinity (K_{D}) for a human NKp46 polypeptide at of less than 10⁻⁸ M, preferably less than 10⁻⁹ M, or preferably less than 10⁻¹⁰M.

Preferably the antigen-binding compound does not lead, directly or indirectly, to the depletion of NK cells expressing NKp46 polypeptides (e.g. do not lead to a 10%, 20%, 50%, 60% or greater elimination or decrease in number of NKp46+ NK cells). Preferably, the antigen-binding compound does not comprise an Fc domain capable of inducing antibody mediated cellular cytoxicity (ADCC) and/or CDC; preferably the antigen-binding compound does not comprise an Fc domain capable of substantially binding to a FcγRIIIA (CD16) polypeptide; preferably the antigen-binding compound lacks an Fc domain (e.g. lacks a CH2 and/or CH3 domain) or comprises an Fc domain of IgG2 or IgG4 isotype; optionally the antigen-binding compound consists of or comprises a Fab, Fab′, Fab′-SH, F (ab′)2, Fv, a diabody, single-chain antibody fragment, or a multispecific antibody comprising multiple different antibody fragments. Preferably the antigen-binding compound is not linked to a toxic moiety. Preferably the antibody does not act as an agonist of the NKp46 polypeptide, e.g. the antibody is preferably not capable of causing cross-linking, in vivo, of NKp46 receptors on an NK cell.

A preferred antigen-binding compound is an isolated antibody, particularly a monoclonal antibody. Fragments and derivatives of such antibodies are also provided. The invention also provides nucleic acids comprising nucleotide sequences encoding such antigen-binding compounds, antibodies; vectors comprising such nucleic acids; host cells and organisms comprising such nucleic acids and/or vectors; and compositions, such as pharmaceutically acceptable compositions and kits, comprising such proteins, nucleic acids, vectors, and/or cells and typically one or more additional ingredients that can be active ingredients or inactive ingredients that promote formulation, delivery, stability, or other characteristics of the composition (e.g., various carriers). The invention further provides various new and useful methods making and using such antigen-binding compounds, antibodies, nucleic acids, vectors, cells, organisms, and/or compositions, such as in the modulation of NK cell activity, for example in the treatment of diseases.

In one aspect, accordingly, the present invention provides a monoclonal antibody or a fragment thereof characterized by:
a) specifically binding to a human NKp46 polypeptide;
b) not specifically binding (e.g., via its Fc domain) to human Fc receptor FeyIIIA (CD16); and
c) when bound to NKp46 on a human NK cell, inhibits NKp46. Preferably the antigen-binding compound inhibits NKp46 signaling in an NK cell. Optionally the antigen-binding compound inhibits binding of a natural ligand of NK46 (e.g. a soluble or immobilized ligand or a ligand expressed on a cell) to an NKp46 polypeptide. Optionally the antigen-binding compound inhibits NKp46-mediated silencing of helios transcription factor in an NK cell (e.g. the antigen-binding compound leads to an increase of helios expression or activity in a developing NKp46-expressing NK cell).

In one embodiment, the antibody inhibits, in a reporter assay, the proliferation of a T cell hybridoma made to express a chimeric protein in which the intracytoplasmic domain of mouse CD3ζ is fused to the extracellular portion of NKp46, when such T cell hybridoma is brought into contact with a NKp46-ligand expressing cell (e.g. a tumor cell which activate the T cell hybridoma in the absence of the test antibody). Optionally the antibody inhibits engagement, by a NKp46 ligand on the NKp46-ligand expressing cell, of the chimeric NKp46 proteins at the T cell surface which would otherwise triggers IL-2 secretion.

In one embodiment, the antibody comprises an Fc domain of human IgG4 isotype.

The present invention thus provides a pharmaceutical composition comprising an anti-NKp46 antibody of the invention, and a pharmaceutically acceptable carrier.

The invention also provides a method for producing an antibody for the treatment of a cancer, infectious disease, transplantation or inflammatory or autoimmune disorder, said method comprising the steps of:
(a) immunizing a non-human mammal with an immunogen comprising a NKp46 polypeptide or providing (e.g. by phage display techniques) a library of antibodies;
(b) selecting an antibody from said immunized mammal or library, wherein said antibody binds and inhibits said NKp46 polypeptide, and
(c) wherein the selected antibody of (b) does not specifically bind (e.g., via its Fc domain) to human Fc receptor FeyIIIA (CD16), or wherein the selected antibody of (b) is further modified such that it does not specifically bind to human Fc receptor FeyIIIA (CD16). Preferably the antigen-binding compound inhibits NKp46 signaling in an NK cell. Optionally the antigen-binding compound inhibits binding of a natural ligand of NK46 (e.g. a soluble or immobilized ligand or a ligand expressed on a cell) to an NKp46 polypeptide. Optionally the antigen-binding compound inhibits NKp46-mediated silencing of helios transcription factor in an NK cell (e.g. the antigen-binding compound leads to an increase of helios expression or activity in a developing NKp46-expressing NK cell, compared to the level observed in the absence of antigen-binding compound). Preferably, an antibody of step (c) will be determined to be suitable for the treatment of a cancer, infectious disease, transplantation or inflammatory or autoimmune disorder.

In one embodiment, provided is a method for the treatment of an autoimmune or inflammatory disease in an individual, comprising:
(a) evaluating the presence, stage and/or evolution of inflammatory or autoimmune disease in an individual; and
(b) administering to said individual an effective dose of a compound that inhibits a NKp46 polypeptide. Optionally, evaluating the presence, stage and/or evolution of disease in an individual comprises analyzing levels of autoantibodies, CRP, or any proteolytic enzyme, inflammatory mediator or marker of ongoing inflammation. If said individual is determined to be suitable for treatment with a compound that inhibits a NKp46 polypeptide (e.g. the individual has arthritis, an exacerbation, etc), administering to said individual an effective dose of a compound that inhibits a NKp46 polypeptide.

In one embodiment, provided is a method for the treatment of an autoimmune or inflammatory disease in an individual, comprising:
(a) determining whether said individual has an established inflammatory or autoimmune disease; and
(b) if said individual has an established inflammatory or autoimmune disease, administering to said patient an effective dose of a compound that inhibits a NKp46 polypeptide.

In one embodiment, provided is a method for the treatment of a cancer, infectious disease, autoimmune or inflammatory disease in an individual, comprising:
(a) determining whether said individual has a cancer, infectious disease, or an autoimmune or inflammatory disease characterized by the presence of T cells capable of being recognized or lysed by an NK cell; and
(b) if said individual has a cancer, infectious disease, autoimmune or inflammatory disease characterized by the presence of T said cells, administering to said patient an effective dose of a compound that inhibits a NKp46 polypeptide.

In one embodiment, the compound that inhibits a NKp46 polypeptide (e.g., an anti-NKp46 antibody) is used in treatment as a single agent (also referred to as monotherapy; the medicament comprising the compound that inhibits a NKp46 polypeptide is free of any other pharmaceutically active agents and/or no additional pharmaceutically active agents are used to treat the individual for the particular disease condition).

In another embodiment, the compound that inhibits a NKp46 polypeptide is administered in combination with a second therapeutic agent, optionally any agent typically used in the context of the particular disease condition.

In one embodiment, the second agent is an agent that induces, via ADCC, the death a cell expressing an antigen to which the second agent binds. In one embodiment the agent is an antibody (e.g. of IgG1 or IgG3 isotype) whose mode of action involves induction of ADCC toward a cell to which the antibody binds. NK cells have an important role in inducing ADCC and increased reactivity of NK cells can be directed to target cells through use of such a second agent. In one embodiment, the second agent is an antibody specific for a cell surface antigens, e.g., membrane antigens. In one embodiment, the antibodies are specific for tumor antigens (e.g., molecules specifically expressed by tumor cells), such as CD20, CD52, ErbB2 (or HER2/Neu), CD33, CD22, CD25, MUC-1, CEA, KDR, αᵥβ₃, etc., particularly lymphoma antigens (e.g., CD20).

In another embodiment, the second agent is an antibody having a constant region of IgG4 isotype or an antibody fragment (e.g. Fab or F(ab)′2 fragment). In another embodiment, the second agent is an antibody linked to a cytotoxic moiety. In one embodiment, the second agent is a non-antibody polypeptide. In one embodiment, the agent is a synthetic small molecule agent. In one embodiment, the agent is a small molecule chemotherapeutic agent. In one embodiment, the agent is a DMARD.

In one embodiment, the second agent is a ligand of an NK cell activating receptor (other than NKp46), e.g. a composition comprising at least a portion of a natural ligand or an antibody that binds and activates an NK cell activating receptor. In one embodiment the agent is an agent that increases the presence of a natural ligand of an NK cell activating receptor on the surface of an target cell (e.g., infected cells, tumor cells, pro-inflammatory cells). NK cell activating receptors include, for example, NKG2D or activating KIR receptors (KIR2DS receptors, KIR2DS2, KIR2DS4), IL-2R, IL-12R, IL-15R, IL-18R and IL-21R. Examples of ligands that act as agonists at activating receptors include, e.g. IL-2, IL-15, IL-21 polypeptides.

The ability of the compounds that inhibit a NKp46 polypeptide to enhance elimination of T cells that may be actively contributing to inflammation makes them suited for use even in chronic settings and established disease, as well as for use in combination with a number of other agents used in inflammatory settings (a second therapeutic agent), in particular agents that decrease inflammation, e.g. such as agents used in chronic and acute settings such as disease modifying anti-rheumatic drugs (DMARDs, e.g. anti-TNFα and MTX) in the case of rheumatoid arthritis and other conditions where such drugs are used. Because mechanisms driving inflammation—particularly acute and chronic—are believed to often be redundant, the antibodies of the invention will be particularly useful for use in combination with agents that act on an inflammation mechanism other than causing direct NK cell killing (e.g., via anti-NKp46 mediated enhancement of NK cell lysis) of T cells, but have a similar biological objective, such as the reduction of pro-inflammatory cytokine production or action, notably the reduction or inhibition of TNFα. In one embodiment of the treatment methods of the invention, compounds that inhibit a NKp46 polypeptide are administered before, concomitantly with or after a second therapeutic agent.

Optionally, in any methods of treatment, the methods further comprise administering to the individual a DMARD. In one embodiment, provided is a method of treating an individual having an autoimmune or inflammatory disease, comprising administering to the individual (a) an effective amount of a compound that inhibits a NKp46 polypeptide, and (b) a DMARD.

Preferably the compound inhibits a NKp46 polypeptide and enhances NK cell reactivity, cytoxicity, activation and/or cytokine production (or enhances the frequency of NK cells having reactivity, cytoxicity, activation and/or cytokine production) as a result of inhibiting said a NKp46 polypeptide over a period of time sufficient to modulate NK cell activity and/or reactivity during NK cell maturation. Preferably the compound comprises an antibody that binds a NKp46 polypeptide, inhibits the function of NKp46 and/or the engagement of NKp46 with a natural ligand of NKp46 (e.g., present on CD11c+ spleen cells). Preferably the antibody further does not comprise an Fc region capable of inducing depletion of an NK cell to which the antibody is bound (e.g. the antibody does not comprise an Fc region capable of binding to CD16).

Anti-NKp46 antibodies can be characterized on the basis of their ability to block or neutralize NKp46-mediated modulation of NK cell reactivity and/or activity during NK cell maturation and thereby enhance NK cell activity against target cells. Preferably the NKp46 antibody inhibits NKp46 signaling in an NK cell. Optionally the antigen-binding compound inhibits binding of a natural ligand of NK46 (e.g. a soluble or immobilized ligand, or a ligand expressed on a cell) to an NKp46 polypeptide. Optionally the antigen-binding compound inhibits NKp46-mediated silencing of hellos transcription factor in an NK cell.

In one aspect, a therapeutically active amount of one or more NKp46 antigen-binding compounds (e.g. antibodies) is an amount of such compounds that results in substantial saturation (at least 50%, 60%, 70%, optionally 75% receptor occupancy) of the NKp46 on NK cells for a period of at least about 1 week, optionally about 2 weeks, optionally about 3 weeks, optionally about one month, following administration of the compound. In one aspect, a therapeutically active amount of one or more NKp46 antigen-binding compounds (e.g. antibodies) is an amount of such compounds that results in substantially complete saturation (at least 80%, 90%, optionally 95% receptor occupancy) of the NKp46 on NK cells for a period of at least about 1 week, optionally about 2 weeks, optionally about 3 weeks, optionally about one month, following administration of the compound. In one embodiment of the methods of the invention, the compound is administered at least two, three, four, five or more times such that the substantial saturation of NKp46 on NK cells is maintained for at least 1, 2, 3, 4, 5 or 6 months.

In one aspect, NKp46 antigen-binding compounds (e.g. antibodies) is dosed in amount and at a frequency that results in substantially complete saturation (90%, optionally 95% receptor occupancy) of the NKp46 on NK cells for a period of at least about 1 week, optionally without a significant “de-saturation” during the treatment period. In one embodiment, a therapeutically active amount of one or more NKp46 antibodies is an amount of such antibody that results in substantially complete NKp46 saturation (90% NKp46 occupancy, optionally 95% NKp46 occupancy) on circulating NK cells for a period of at least about 2 weeks, optionally about 3 weeks, optionally about one month, following administration of the antibody, and the antibody is dosed at least twice, wherein dosing occurs about once every 2 weeks, once every 3 weeks, or once per month (subsequent doses are separated by about 2 weeks, 3 weeks or one month). In one embodiment of the methods of the invention, the compound is administered at least two, three, four, five or more times such that the substantially complete NKp46 saturation on NK cells is maintained for at least 1, 2, 3, 4, 5 or 6 months.

In another aspect, the NKp46 antigen-binding compound is dosed in amount and at a frequency that results in substantial or substantially complete saturation of NKp46 on NK cells for a period of at least about 1 week, 2 weeks, 3 weeks or one month, and that permits a significant “de-saturation” during the treatment period prior to the subsequent administration of anti-NKp46 antibody. Since anti-NKp46 antibodies may inhibit the ability of NK cells to recognize and lyse target cells via their NKp46 polypeptides, such de-saturation may permit maximal activity of the hyper-reactive NK cells that have developed during the period in which the NK cells' NKp46 was blocked by the anti-NKp46 antibody. In one embodiment, a therapeutically active amount of one or more anti-NKp46 antibodies is an amount of such antibody that results in substantial or substantially complete NKp46 saturation on circulating NK cells for a period of at least about 1 week, 2 weeks, optionally about 3 weeks, optionally about one month, following administration of the antibody, and the antibody is dosed at least twice, wherein dosing occurs at least about once every two months (subsequent doses are separated by about two months or more than two months).

These aspects are more fully described in, and additional aspects, features, and advantages of the invention will be apparent from, the description of the invention provided herein.

### BRIEF DESCRIPTION OF THE DRAWINGS

FIG. 1. Noé mice are more resistant to MCMV because of hyperresponsive NK cells

(A) Frequencies of IFN-γ-producing and CD107a⁺ IL-2 activated WT and Noé NK cells after co-culture with YAC tumor targets. Unpaired t test; n=9-10, P<0.0001 and P=0.0003, Mann-Whitney test.

(B) Kaplan-Meier representation of WT (closed symbols) and Noé (open symbols) mice survival after MCMV infection with 5,300 PFU/gram of body weight. n=15-20, P<0.0001, Log-rank Mantel-Cox test.

(C) WT and Noé mice were treated with an anti-NK1.1 antibody and infected the following day with MCMV. Data show Kaplan-Meier representation of WT (closed symbols) and Noé (open symbols) mice survival after MCMV infection with 5,300 PFU/gBW. n=15-20.

(D) Measure of MCMV viral load in spleen and liver of WT and Noé mice 4 days post-infection. Dotted lines indicate limit of detection. n=10, **P=0.0079 and *P=0.0112, Mann-Whitney test.

(E) Frequencies of IFN-γ-producing WT and Noé NK cells after 1.5 days of MCMV infection in spleen. n=5, **P=0.0079 and *P=0.0159, Mann-Whitney test.

FIG. 2. A point mutation in Ncr1/NKp46 gene is responsible for the hyperresponsiveness of Noé NK cells

(A) Representation of the Ncr1 gene. The W32R mutation is indicated.

(B-C) Superposition of the domain 1 of WT (B) and W32R mutant NKp46 protein (C).

(D) Flow cytometric analysis of WT and Noé splenocytes stained with anti-NK1.1 and anti-NKp46 antibodies. Results are representative of the Noé phenotype.

(E) Flow cytometric measurement of mouse and human NKp46 surface expression (empty histogram) or isotypes control (filled hystograms) gated on NK1.1⁺CD3⁻ splenocytes from WT, Ncr1^{Noé/Noé} and Ncr1^{Noé/Noé}×huNKp46 Tg littermates. Results are representative of at least 20 animals.

(F) Frequencies of IFN-γ-producing and CD107a⁺ in IL-2 activated WT, Ncr1^{Noé/Noé} and Ncr1^{Noé/Noé}×huNKp46 Tg NK cells after co-culture with YAC tumor targets. n=6-8, Kruskal-Wallis test with Dunns comparison post-test.

(G) Kaplan-Meier representation of WT (closed circles), Ncr1^{Noé/Noé} (open circles) and Ncr1^{Noé/Noé}×huNKp46 Tg (open triangles) mice survival after MCMV infection with 5,300 PFU/gBW. n=8-10, P<0.0001, Log-rank Mantel Cox test.

FIG. 3. NKp46-triggering controls Helios silencing to set NK cell responsiveness

(A-D) Flow cytometry analysis of CD11b expression in NK1.1⁺CD3⁻ bone marrow from (A) WT and Noé mice (P=0.0006), (B) mixed bone marrow chimera with CD45.1⁺ WT and CD45.2⁺ Ncr1^{Noé/Noé} cells (P=0.0212), (C) NK cell depleted NDE mice treated with the anti-NKp46 mAb or an isotype control of 15 days (P=0.008), and (D) WT, Ncr1^{Noé/Noé} and Ncr1^{Noé/Noé}×huNKP46 Tg littermates, n=6-8, (A-C), Mann-Whitney test, (D) Kruskal-Wallis test with Dunn's comparisons post-test.

(E-G) Helios transcripts were quantified by real-time PCR (E) in sorted CD11b⁻ and CD11b⁺ bone marrow NK from WT mice, (F) in CD11b⁺ NK cells from WT, Ncr1^{Noé/Noé} and Ncr1^{Noé/Noé}×huNKp46 Tg mice, (G) in CD11b⁺ NK cells from NK cell-depleted NDE mice treated with anti-NKp46 mAb or an isoytpe control for 15 days. Results were normalized with respect to Gapdh (glyceraldehyde phosphate dehydrogenase) and expressed as arbitrary units. Data result from a pool of 2 independent experiments with a total of 6 animals per group.

FIG. 4. Hyperresponsive NK cells limit anti-viral T cell immunity

Panels A-F relate to infection of mice with MCMV at a dose of 1,600 PFU/gram of body weight.

(A) Frequencies of MCMV-specific CD8⁺ T cells among total CD8⁺ T cells were measured by a H2D^{b}/m45 pentamer staining in spleen of WT (closed symbols) and Ncr1^{Noé/Noé} (open symbols) mice at indicated time points post-infection. n=5-7, P**<0.001, Two-way Anova test with Bonferroni comparison post-test.

(B) Absolute numbers of total CD8⁺ T cells and H2D^{b}/m45⁺ CD8⁺ T cells in spleen of WT and Ncr1^{Noé/Noé} mice 7 days post infection. n=7, P=0.0021, Mann-Whitney test.

(C-D) Ex vivo restimulation of (C) spleen H2D^{b}/m45-specific CD8⁺ T cells (D) liver CD4⁺ T cells. Frequencies of IFN-γ-producing cells among total CD8⁺ T cells or CD4⁺ T cells are shown. N=5-7, **P<0.01 and ***P<0.001, Two-way Anova test with Bonferroni comparison post-test.

(E) Frequencies of H2D^{b}/m45⁺CD8⁺ T cells among total CD8⁺ T cells in spleen of WT, Ncr1^{Noé/Noé} and Ncr1^{Noé/Noé}×huNKp46 Tg littermates 7 days post infection. n=5.

(F-G) Ex vivo restimulation of (F) spleen H2D^{b}/m45-specific CD8⁺ T cells or (G) liver CD4⁺ T cells. Frequencies of IFN-γ-producing cells are shown for WT, Ncr1^{Noé/Noé} and Ncr1^{Noé/Noé} huNKp46 Tg mice 7 days post infection. n=5. **P<0.01 and ***P<0.001, Kruskal-Wallis test with Dunn's comparison post-test.

Panels H-J relate to infection with Listeria monocytogenes (Lm).

(H-J) CD8⁺ T-cell protective immunity generated in response to intracellular bacteria Listeria monocytogenes (Lm) expressing ovalbumin (OVA) was analyzed. The frequencies of IFN-γ-producing NK cells were 36%±3.2% higher in Ncr1^{Noé/Noé} mice than in the WT 24 hours after infection with Lm-OVA (FIG. 3H; P=0.0119). Following primary Lm-OVA infection and rechallenge 30 days later, the percentages of memory Lm-OVA-specific CD8⁺ T cells capable to produce IFN-γ were 35%±4.4% and 36%±4% lower in Ncr1^{Noé/Noé} mice than in WT mice and Ncr1^{Noé/Noé}huNKKp46 Tg mice, respectively (FIG. 3I; P<0.01 and P<0.05, respectively). This alteration in the quality of Lm-OVA-specific CD8⁺ memory T cells in Ncr1^{Noé/Noé} mice was associated with a bacterial load in the spleen 12 times higher than that in WT mice and Ncr1^{Noé/Noé}huNKp46 Tg littermates (FIG. 3J; P<0.001 and P<0.01, respectively).

FIG. 5.

(A) WT (closed symbols) and Noé (open symbols) spleen cell suspensions were stimulated with ani-NK.1-coated antibody and the frequencies of IFN-γ-producing cells among NK cells were determined by flow cytometry.

(B) Representative FACS profiles of WT and Noé spleen cells stained with anti-NK1.1 and anti-CD3 antibodies.

(C) WT (closed symbols) and Noé (open symbols) spleen cell suspensions were stimulated with ani-NK.1 antibody coated plates in the presence of indicated concentration of wortmannin. The frequencies of IFN-γ-producing cells among NK cells were determined by flow cytometry. Results obtained with untreated control cells were considered as 100%. In (A) and (C), n=6, **P<0.01, ***P<0.001, Two-way Anova test with Bonferroni post-test.

FIG. 6. The Noé is NK cell intrinsic.

(A) Schematic representation of the experiment, a 1:1 mix of CD45.1⁺ WT and CD45.2⁺ Noé bone marrow cells was used to reconstitute lethally irradiated recipients. 8 weeks later, splenic NK cells were analyzed.

(B) Frequencies of IFN-γ-producing and CD107a⁺ NK cells were analyzed in IL-2-activated NK cells from CD45.1⁺ WT and CD45.2⁺ Noé cells. After co-culture with YAC-1 tumor targets. N=8, **P=0.0038 and ***P=0.0002, Mann-Whitney test.

FIG. 7. Noé mice are more resistant to MCMV infection.

Kaplan-Maier representation of WT (closed symbols) and Noé (open symbols) mice survival after MCMV infection with 1,600 (A) and 7,500 (B) PFU/gram of body weight (gBW). n=5. P=0.0076, Log-rand Mantel Cox test.

FIG. 8. Similar numbers of NK cells in Noé and WT mice during MCMV infection.

(A) Absolute numbers of total NK1.1⁺CD3⁻ NK cells in spleen of WT (closed symbols) and Noé (open symbols) mice at the indicated time points after MCMV infection.

(B) Absolute numbers of total Ly49H⁺ NK1.1⁺CD3⁻ NK cells among the total NK cell population in the spleen of WT (closed symbols) and Noé (open symbols) mice at the indicated time points after MCMV infection.

FIG. 9. Mutations identified in Noé mice after whole genome sequencing.

Homozygous mutations identified by resequencing the genome of a Noé mouse as compared to a WT reference. (A) Non-synonymous homozygous mutations in coding regions. (B) Homozygous mutations in splice sites. Genes expressed in NK cells are indicated (*).

FIG. 10. Normal level of Ncr1 transcripts in Noé NK cells.

Relative expression of Ncr1 transcripts in sorted CD11b⁻ (A) and CD11b⁺ (B) NK cells from spleen of WT and Noé mice. Results were normalized with respect to Gapdh and expressed as arbitrary units. Data result from a pool of 2 independent experiments.

FIG. 11. Noé NK cells are not stained with anti-NKp46 polyserum.

Flow cytometric analysis of NKp46 expression on splenic WT and Noé NK1.1⁺ CD3⁻ NK cells using an anti-NKp46 polyserum or control serum. Results are representative of at least 10 animals.

FIG. 12. Neutralizing NKp46 during NK cell development induces a Noé-like phenotype in WT mice.

(A) Schematic representation of the experiment. NDE mice were treated with DT to deplete NK cells. Upon reconstitution of the NK cell compartment, mice were treated with anti-NKp46 or control monoclonal antibodies every 2-3 days for 11 days. Spleen cells from anti-NKp46 and control treated mice were analyzed at day 15.

(B) Flow cytometric staining of NK1.1⁺CD3⁻ NK cells with the anti-NKp46 antibody coupled Alexa647 (left panels) or with a secondary anti-rat antibody (right panels) (empty histograms). Control stainings are also shown (filled histograms). Results are representative of at least 5 animals.

(C) Frequencies of IFN-γ-producing and CD107a⁺ in IL-2 activated NK cells after a co-culture with YAC-1 tumor targets. N=8, P=0.0009, Mann-Whitney test.

FIG. 13. CD11b⁻ and CD11b⁺ Ncr1^{Noé/Noé} NK cells are hyper-responsive.

WT (closed symbols) and Ncr1^{Noé/Noé} (open symbols) spleen cell suspensions were stimulated with anti-NK1.1 antibody-coated plates. The frequencies of IFN-γ-producing cells among CD11b⁻ (A) and CD11b⁺ (B) NK cells were determined by flow cytometry. N=6, *P=0.0124, **P=0.005, Mann-Whitney test.

FIG. 14. mRNA levels of Ikaros transcription factor family in NK cells.

CD11b⁻ and mature CD11b⁺ splenic NK cells were analysed using pan-genomic transcriptomic analysis (Chiossone et al (2009) blood 113: 5488). FIG. 14 shows the mRNA levels of Ikaros transcription factor family, where Helios mRNA is decreased in CD11b⁺ cells while other transcription factors remain at similar levels.

FIGS. 15 and 16: Identification of NKp46 blocking antibodies

FIG. 15, panel A, shows DOMSP30 reporter cells are activated (as expressed by IL-2 induced CTLL2 cell proliferation) when brought into contact with Hela EV2 cells, and that addition of anti-NKp30 antibodies (clone AZ20) reduced CTLL-2 cell proliferation indicating that the antibodies blocked NKp30. FIG. 15, panel B, shows DOMSP46 reporter cells are not activated when brought into contact with Hela EV2 cells.

FIG. 16, panel B, shows DOMSP46 reporter cells were activated when brought into contact with B12 cells, showing that B12 cells express a NKp46 ligand. Addition of anti-NKp46 antibodies (clone Bab281) reduced CTLL-2 cell proliferation indicating that the antibodies blocked NKp46. Antibodies to NKp30 (AZ20) did not reduce CTLL-2 cell proliferation. FIG. 16, panel A, shows DOMSP30 reporter cells are not activated when brought into contact with B12 cells.

FIG. 17: Long-term NKp46 blockade enhances NK cell responsiveness

Panel A shows the administration scheme used to modify the responsiveness of NK cells by injecting anti-NKp46 mAb, at steady state, in WT mice. Panel B shows that in vivo blockade of NKp46 by the mAb for 13 days was sufficient to enhance NK cell responsiveness (*P=0.03 and **P=0.0081).

FIG. 18: Short-term NKp46 blockade does not increase the reactivity of NK cells

Panel A shows the administration scheme used. Panels B and E show flow cytometry staining of NK1.1+Cd3− NK cells with the anti-NKp46 coupled to Alexa 647 or with a secondary anti-rat antibody (empty histograms). Control stainings are also shown (filled histograms). Panels C and F show frequencies of IFN-γ-producing and CD107a+ IL-2 activated NK cells after a co-culture with YAC-1 tumor targets. Short treatments lasting 24 to 72 hours, were sufficient to saturate NKp46 receptors but did not increase the reactivity of NK cells in response to YAC-1 tumor targets.

### DETAILED DESCRIPTION OF THE INVENTION

### Introduction

The present invention provides novel methods for producing and using antibodies and particularly NKp46-modulating antibodies suitable for the prophylaxis and treatment of disorders such as cancer, infection, autoimmunity, inflammation and transplantation. Antibodies, antibody derivatives, antibody fragments, and cells producing them are encompassed, as are methods of producing the same and methods of treating or diagnosing patients using the antibodies and compounds.

### DEFINITIONS

As used herein, “NK cells” refers to a sub-population of lymphocytes that is involved in non-conventional immunity. NK cells can be identified by virtue of certain characteristics and biological properties, such as the expression of specific surface antigens including CD56 and/or CD16 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context of this invention “active” NK cells designate biologically active NK cells, including NK cells having the capacity of lysing target cells or enhancing the immune function of other cells. For instance, an “active” NK cell can be able to kill cells that express a ligand for an activating NK receptor and/or fail to express MHC/HLA antigens recognized by a KIR on the NK cell. NK cells can be obtained by various techniques known in the art, such as isolation from blood samples, cytapheresis, tissue or cell collections, etc. Useful protocols for assays involving NK cells can be found in Natural Killer Cells Protocols (edited by Campbell K S and Colonna M). Human Press. pp. 219-238 (2000).

As used herein, “T cells” refers to a sub-population of lymphocytes that mature in the thymus, and which display, among other molecules T cell receptors on their surface. T cells can be identified by virtue of certain characteristics and biological properties, such as the expression of specific surface antigens including the TCR, CD4 or CD8, the ability of certain

T cells to kill tumor or infected cells, the ability of certain T cells to activate other cells of the immune system, and the ability to release protein molecules called cytokines that stimulate or inhibit the immune response. Any of these characteristics and activities can be used to identify T cells, using methods well known in the art. Within the context of this invention, “active” or “activated” T cells designate biologically active T cells, more particularly T cells having the capacity of cytolysis or of stimulating an immune response by, e.g., secreting cytokines. Active cells can be detected in any of a number of well known methods, including functional assays and expression-based assays such as the expression of cytokines such as TNF-alpha.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG and/or IgM are the preferred classes of antibodies employed in this invention, with IgG being particularly preferred, because they are the most common antibodies in the physiological situation and because they are most easily made in a laboratory setting. Preferably the antibody of this invention is a monoclonal antibody. Particularly preferred are humanized, chimeric, human, or otherwise-human-suitable antibodies. “Antibodies” also includes any fragment or derivative of any of the herein described antibodies.

“Specific binding” or “specificity” refers to the ability of an antibody or other agent to detectably bind an epitope presented on an antigen, such as a NKp46, while having relatively little detectable reactivity with non-NKp46 proteins or structures (such as other proteins presented on NK cells, or on other cell types). Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a NKp46 polypeptide).

The terms “depleting”, with respect to NKp46-expressing cells means a process, method, or compound that can kill, eliminate, lyse or induce such killing, elimination or lysis, so as to negatively affect the number of NKp46-expressing cells present in a sample or in a subject.

When an antibody is said to “compete with” a particular monoclonal antibody (e.g. Bab281, 9E2 or 195314), it means that the antibody competes with the monoclonal antibody in a binding assay using either recombinant NKp46 molecules or surface expressed NKp46 molecules. For example, if a test antibody reduces the binding of Bab281, 9E2 or 195314 to a NKp46 polypeptide or NKp46-expressing cell in a binding assay, the antibody is said to “compete” respectively with Bab281, 9E2 or 195314.

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Kₐ is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

Within the context of this invention a “determinant” designates a site of interaction or binding on a polypeptide.

The term “epitope” is defined as an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

By “immunogenic fragment,” it is herein meant any polypeptidic or peptidic fragment that is capable of eliciting an immune response such as (i) the generation of antibodies binding said fragment and/or binding any form of the molecule comprising said fragment, including the membrane-bound receptor and mutants derived therefrom, (ii) the stimulation of a T-cell response involving T-cells reacting to the bi-molecular complex comprising any MHC molecule and a peptide derived from said fragment, (iii) the binding of transfected vehicles such as bacteriophages or bacteria expressing genes encoding mammalian immunoglobulins. Alternatively, an immunogenic fragment also refers to any construction capable of eliciting an immune response as defined above, such as a peptidic fragment conjugated to a carrier protein by covalent coupling, a chimeric recombinant polypeptide construct comprising said peptidic fragment in its amino acid sequence, and specifically includes cells transfected with a cDNA of which sequence comprises a portion encoding said fragment.

For the purposes of the present invention, a “humanized” or “human” antibody refers to an antibody in which the constant and variable framework region of one or more human immunoglobulins is fused with the binding region, e.g. the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet. 7:13; Lonberg et al. (1994) Nature 368:856; Taylor et al. (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated in their entirety by reference).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, Md.).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

### Producing Anti-NKp46 Antibodies

The antibodies of this invention specifically bind NKp46, preferably the extracellular domain of NKp46. Antibodies of the invention furthermore inhibit NKp46 function. Antibodies of the invention are furthermore capable of inhibiting the NKp46 signaling pathway and are optionally furthermore capable of down-modulating the silencing of the Helios transcription factor mediated by NKp46 engagement in NK cells. The ability of the inhibitory antibodies to specifically inhibit NKp46 signaling pathway and to thereby modify tuning of NK cells during maturation so as to yield hyper-reactive NK cells makes them useful for numerous applications, in particular for treating or preventing diseases where increasing the activity of NK cells is desirable.

“NKp46 polypeptide” and “NKp46 receptor” refer to a protein or polypeptide encoded by the Ncr1 gene or by a cDNA prepared from such a gene. Any naturally occurring isoform, allele or variant is encompassed by the term NKp46 polypeptide (e.g., an NKp46 polypeptide 90%, 95%, 98% or 99% identical to SEQ ID NO 1, or a contiguous sequence of at least 20, 30, 50, 100 or 200 amino acid residues thereof). The 304 amino acid residue sequence of human NKp46 (isoform a) is shown as follows:

(SEQ ID NO: 1)
MSSTLPALLC VGLCLSQRIS AQQQTLPKPF IWAEPEFMVP
KEKQVTICCQ GNYGAVEYQL HFEGSLFAVD RPKPPERINK
VKFYIPDMNS RMAGQYSCIY RVGELWSEPS NLLDLVVTEM
YDTPTLSVHP GPEVISGEKV TFYCRLDTAT SMFLLLKEGR
SSHVQRGYGK VQAEFPLGPV TTAHRGTYRC FGSYNNHAWS
FPSEPVKLLV TGDIENTSLA PEDPTFPADT WGTYLLTTET
GLQKDHALWD HTAQNLLRMG LAFLVLVALV WFLVEDWLSR
KRTRERASRA STWEGRRRLN TQTL.

SEQ ID NO: 1 corresponds to NCBI accession number NP_{—}004820, the disclosure of which is incorporated herein by reference. The human NKp46 mRNA sequence is described in NCBI accession number NM_{—}004829, the disclosure of which is incorporated herein by reference.

Examples of antibodies that inhibit human NKp46 include, e.g, Bab281, mIgG1, available commercially from Beckman Coulter, Inc. (Brea, Calif., USA) (see Pessino et al, J. Exp. Med, 1998, 188 (5): 953-960 and Sivori et al, Eur J Immunol, 1999. 29:1656-1666) describing chromium release cytotoxicity assays). Another NKp46 blocking antibody is 9E2, mIgG1, available commercially from Becton Dickinson (Franklin Lakes, N.J., USA) and Miltenyi Biotec (Bergisch Gladback, Germany) (see Brando et al, (2005) J. Leukoc. Biol. 78:359-371; and E1-Sherbiny et al, (2007) Cancer Research 67(18):8444-9). Another anti-NKp46 blocking antibody is 195314, mlgG2b, available commercially from R&D Systems, Inc. (Minneapolis, USA) (see Nolte-'t Hoen et al, (2007) Blood 109:670-673). These antibodies all bind human NKp46, are of murine origin and have murine Fc domains. The anti-NKp46 antibodies of the invention may include antibodies having variable region or CDR sequences from such Bab281, 9E2 or 195314 antibodies (e.g. a heavy and/or light chain variable region fused to a human constant region; a heavy chain variable region fused to a human IgG4 heavy chain constant region); alternatively, the anti-NKp46 antibodies of the invention may be an antibody other than the antibodies having variable region or CDR sequences from a Bab281, 9E2 or 195314 antibody.

In one aspect, the invention provides an antibody that competes with monoclonal antibody BAB281, 9E2 or 195314 and recognizes, binds to, or has immunospecificity for substantially or essentially the same, or the same, epitope or “epitopic site” on a NKp46 molecule as monoclonal antibody Bab281, 9E2 or 195314. In other embodiments, the monoclonal antibody consists of, or is a derivative or fragment of, antibody Bab281, 9E2 or 195314.

It will be appreciated that, while preferred antibodies bind to the same epitope as antibody Bab281, 9E2 or 195314, the present antibodies can recognize and be raised against any part of the NKp46 polypeptide so long as the antibody inhibits NKp46 signalling in NK cells. For example, any fragment of NKp46, preferably but not exclusively human NKp46, or any combination of NKp46 fragments, can be used as immunogens to raise antibodies, and the antibodies of the invention can recognize epitopes at any location within the NKp46 polypeptide, so long as they can do so on NKp46 expressing NK cells as described herein. In an embodiment, the recognized epitopes are present on the cell surface, i.e. they are accessible to antibodies present outside of the cell. Most preferably, the epitope is the epitope specifically recognized by antibody Bab281, 9E2 or 195314. Further, antibodies recognizing distinct epitopes within NKp46 can be used in combination, e.g. to bind to NKp46 polypeptides with maximum efficacy and breadth among different individuals.

The antibodies of this invention may be produced by a variety of techniques known in the art. Typically, they are produced by immunization of a non-human animal, preferably a mouse, with an immunogen comprising a NKp46 polypeptide, preferably a human NKp46 polypeptide. The NKp46 polypeptide may comprise the full length sequence of a human NKp46 polypeptide, or a fragment or derivative thereof, typically an immunogenic fragment, i.e., a portion of the polypeptide comprising an epitope exposed on the surface of cells expressing a NKp46 polypeptide, preferably the epitope recognized by the Bab281, 9E2 or 195314 antibody. Such fragments typically contain at least about 7 consecutive amino acids of the mature polypeptide sequence, even more preferably at least about 10 consecutive amino acids thereof. Fragments typically are essentially derived from the extra-cellular domain of the receptor. In a preferred embodiment, the immunogen comprises a wild-type human NKp46 polypeptide in a lipid membrane, typically at the surface of a cell. In a specific embodiment, the immunogen comprises intact cells, particularly intact human cells, optionally treated or lysed. In another preferred embodiment, the polypeptide is a recombinant NKp46 polypeptide.

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, N.Y. (1988), the entire disclosure of which is herein incorporated by reference). The immunogen is suspended or dissolved in a buffer, optionally with an adjuvant, such as complete or incomplete Freund's adjuvant. Methods for determining the amount of immunogen, types of buffers and amounts of adjuvant are well known to those of skill in the art and are not limiting in any way on the present invention. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with an adjuvant such as incomplete Freund's adjuvant. The recall injections are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be used as long as they result in the production of B cells expressing an antibody directed to the antigen used in immunization.

For polyclonal antibody preparation, serum is obtained from an immunized non-human animal and the antibodies present therein isolated by well-known techniques. The serum may be affinity purified using any of the immunogens set forth above linked to a solid support so as to obtain antibodies that react with NKp46 polypeptides.

In an alternate embodiment, lymphocytes from a non-immunized non-human mammal are isolated, grown in vitro, and then exposed to the immunogen in cell culture. The lymphocytes are then harvested and the fusion step described below is carried out.

For preferred monoclonal antibodies, the next step is the isolation of splenocytes from the immunized non-human mammal and the subsequent fusion of those splenocytes with an immortalized cell in order to form an antibody-producing hybridoma. The isolation of splenocytes from a non-human mammal is well-known in the art and typically involves removing the spleen from an anesthetized non-human mammal, cutting it into small pieces and squeezing the splenocytes from the splenic capsule through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer.

Once isolated and present in single cell suspension, the lymphocytes can be fused to an immortal cell line. This is typically a mouse myeloma cell line, although many other immortal cell lines useful for creating hybridomas are known in the art. Preferred murine myeloma lines include, but are not limited to, those derived from MOPC-21 and MPC-11 mouse tumors available from the Salk Institute Cell Distribution Center, San Diego, U.S.A., X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Hybridomas are typically grown on a feeder layer of macrophages. The macrophages are preferably from littermates of the non-human mammal used to isolate splenocytes and are typically primed with incomplete Freund's adjuvant or the like several days before plating the hybridomas. Fusion methods are described in Goding, “Monoclonal Antibodies: Principles and Practice,” pp. 59-103 (Academic Press, 1986), the disclosure of which is herein incorporated by reference.

The cells are allowed to grow in the selection media for sufficient time for colony formation and antibody production. This is usually between about 7 and about 14 days.

The hybridoma colonies are then assayed for the production of antibodies that specifically bind to NKp46 polypeptide gene products, optionally the epitope specifically recognized by antibody Bab281, 9E2 or 195314. The assay is typically a colorimetric ELISA-type assay, although any assay may be employed that can be adapted to the wells that the hybridomas are grown in. Other assays include radioimmunoassays or fluorescence activated cell sorting. The wells positive for the desired antibody production are examined to determine if one or more distinct colonies are present. If more than one colony is present, the cells may be re-cloned and grown to ensure that only a single cell has given rise to the colony producing the desired antibody. Typically, the antibodies will also be tested for the ability to bind to NKp46 polypeptides, e.g., NKp46-expressing cells.

Hybridomas that are confirmed to produce a monoclonal antibody of this invention can be grown up in larger amounts in an appropriate medium, such as DMEM or RPMI-1640. Alternatively, the hybridoma cells can be grown in vivo as ascites tumors in an animal. After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Biosciences, publication No. 18-1037-46, Edition AC, the disclosure of which is hereby incorporated by reference). The bound antibody is typically eluted from protein A/protein G columns by using low pH buffers (glycine or acetate buffers of pH 3.0 or less) with immediate neutralization of antibody-containing fractions. These fractions are pooled, dialyzed, and concentrated as needed.

Positive wells with a single apparent colony are typically re-cloned and re-assayed to insure only one monoclonal antibody is being detected and produced.

Antibodies may also be produced by selection of combinatorial libraries of immunoglobulins, as disclosed for instance in (Ward et al. Nature, 341 (1989) p. 544, the entire disclosure of which is herein incorporated by reference).

The identification of one or more antibodies that bind(s) to NKp46, particularly substantially or essentially the same epitope as monoclonal antibody Bab281, 9E2 or 195314, can be readily determined using any one of a variety of immunological screening assays in which antibody competition can be assessed. Many such assays are routinely practiced and are well known in the art (see, e.g., U.S. Pat. No. 5,660,827, issued Aug. 26, 1997, which is specifically incorporated herein by reference). It will be understood that actually determining the epitope to which an antibody described herein binds is not in any way required to identify an antibody that binds to the same or substantially the same epitope as the monoclonal antibody described herein.

For example, where the test antibodies to be examined are obtained from different source animals, or are even of a different Ig isotype, a simple competition assay may be employed in which the control (Bab281, 9E2 or 195314, for example) and test antibodies are admixed (or pre-adsorbed) and applied to a sample containing NKp46 polypeptides. Protocols based upon western blotting and the use of BIACORE analysis are suitable for use in such competition studies.

In certain embodiments, one pre-mixes the control antibodies (Bab281, 9E2 or 195314, for example) with varying amounts of the test antibodies (e.g., about 1:10 or about 1:100) for a period of time prior to applying to the NKp46 antigen sample. In other embodiments, the control and varying amounts of test antibodies can simply be admixed during exposure to the NKp46 antigen sample. As long as one can distinguish bound from free antibodies (e.g., by using separation or washing techniques to eliminate unbound antibodies) and Bab281, 9E2 or 195314 from the test antibodies (e.g., by using species-specific or isotype-specific secondary antibodies or by specifically labeling Bab281, 9E2 or 195314 with a detectable label) one can determine if the test antibodies reduce the binding of Bab281, 9E2 or 195314 to the antigens, indicating that the test antibody recognizes substantially the same epitope as Bab281, 9E2 or 195314. The binding of the (labeled) control antibodies in the absence of a completely irrelevant antibody can serve as the control high value. The control low value can be obtained by incubating the labeled (Bab281, 9E2 or 195314) antibodies with unlabelled antibodies of exactly the same type (Bab281, 9E2 or 195314), where competition would occur and reduce binding of the labeled antibodies. In a test assay, a significant reduction in labeled antibody reactivity in the presence of a test antibody is indicative of a test antibody that recognizes substantially the same epitope, i.e., one that “cross-reacts” or competes with the labeled (Bab281, 9E2 or 195314) antibody. Any test antibody that reduces the binding of Bab281, 9E2 or 195314 to NKp46 antigens by at least about 50%, such as at least about 60%, or more preferably at least about 80% or 90% (e.g., about 65-100%), at any ratio of Bab281, 9E2 or 195314:test antibody between about 1:10 and about 1:100 is considered to be an antibody that binds to substantially the same epitope or determinant as Bab281, 9E2 or 195314. Preferably, such test antibody will reduce the binding of Bab281, 9E2 or 195314 to the NKp46 antigen by at least about 90% (e.g., about 95%).

Competition can also be assessed by, for example, a flow cytometry test. In such a test, cells bearing a given NKp46 polypeptide can be incubated first with Bab281, 9E2 or 195314, for example, and then with the test antibody labeled with a fluorochrome or biotin. The antibody is said to compete with Bab281, 9E2 or 195314 if the binding obtained upon preincubation with a saturating amount of Bab281, 9E2 or 195314 is about 80%, preferably about 50%, about 40% or less (e.g., about 30%, 20% or 10%) of the binding (as measured by mean of fluorescence) obtained by the antibody without preincubation with Bab281, 9E2 or 195314. Alternatively, an antibody is said to compete with Bab281, 9E2 or 195314 if the binding obtained with a labeled Bab281, 9E2 or 195314 antibody (by a fluorochrome or biotin) on cells preincubated with a saturating amount of test antibody is about 80%, preferably about 50%, about 40%, or less (e.g., about 30%, 20% or 10%) of the binding obtained without preincubation with the test antibody.

A simple competition assay in which a test antibody is pre-adsorbed and applied at saturating concentration to a surface onto which a NKp46 antigen is immobilized may also be employed. The surface in the simple competition assay is preferably a BIACORE chip (or other media suitable for surface plasmon resonance analysis). The control antibody (e.g., Bab281, 9E2 or 195314) is then brought into contact with the surface at a NKp46-saturating concentration and the NKp46 and surface binding of the control antibody is measured. This binding of the control antibody is compared with the binding of the control antibody to the NKp46-containing surface in the absence of test antibody. In a test assay, a significant reduction in binding of the NKp46-containing surface by the control antibody in the presence of a test antibody indicates that the test antibody recognizes substantially the same epitope as the control antibody such that the test antibody “cross-reacts” with the control antibody. Any test antibody that reduces the binding of control (such as Bab281, 9E2 or 195314) antibody to a NKp46 antigen by at least about 30% or more, preferably about 40%, can be considered to be an antibody that binds to substantially the same epitope or determinant as a control (e.g., Bab281, 9E2 or 195314). Preferably, such a test antibody will reduce the binding of the control antibody (e.g., Bab281, 9E2 or 195314) to the NKp46 antigen by at least about 50% (e.g., at least about 60%, at least about 70%, or more). It will be appreciated that the order of control and test antibodies can be reversed: that is, the control antibody can be first bound to the surface and the test antibody is brought into contact with the surface thereafter in a competition assay. Preferably, the antibody having higher affinity for the NKp46 antigen is bound to the surface first, as it will be expected that the decrease in binding seen for the second antibody (assuming the antibodies are cross-reacting) will be of greater magnitude. Further examples of such assays are provided in, e.g., Saunal (1995) J. Immunol. Methods 183: 33-41, the disclosure of which is incorporated herein by reference.

Determination of whether an antibody binds within an epitope region can be carried out in ways known to the person skilled in the art. As one example of such mapping/characterization methods, an epitope region for an anti-NKp46 antibody may be determined by epitope “foot-printing” using chemical modification of the exposed amines/carboxyls in the NKp46 protein. One specific example of such a foot-printing technique is the use of HXMS (hydrogen-deuterium exchange detected by mass spectrometry) wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry. See, e.g., Ehring H, Analytical Biochemistry, Vol. 267 (2) pp. 252-259 (1999) Engen, J. R. and Smith, D. L. (2001) Anal. Chem. 73, 256A-265A. Another example of a suitable epitope identification technique is nuclear magnetic resonance epitope mapping (NMR), where typically the position of the signals in two-dimensional NMR spectra of the free antigen and the antigen complexed with the antigen binding peptide, such as an antibody, are compared. The antigen typically is selectively isotopically labeled with 15N so that only signals corresponding to the antigen and no signals from the antigen binding peptide are seen in the NMR-spectrum. Antigen signals originating from amino acids involved in the interaction with the antigen binding peptide typically will shift position in the spectrum of the complex compared to the spectrum of the free antigen, and the amino acids involved in the binding can be identified that way. See, e.g., Ernst Schering Res Found Workshop. 2004; (44): 149-67; Huang et Journal of Molecular Biology, Vol. 281 (1) pp. 61-67 (1998); and Saito and Patterson, Methods. 1996 June; 9 (3): 516-24.

Epitope mapping/characterization also can be performed using mass spectrometry methods. See, e.g., Downward, J Mass Spectrom. 2000 April; 35 (4): 493-503 and Kiselar and Downard, Anal Chem. 1999 May 1; 71 (9): 1792-801. Protease digestion techniques also can be useful in the context of epitope mapping and identification. Antigenic determinant-relevant regions/sequences can be determined by protease digestion, e.g. by using trypsin in a ratio of about 1:50 to NKp46 or o/n digestion at and pH 7-8, followed by mass spectrometry (MS) analysis for peptide identification. The peptides protected from trypsin cleavage by the anti-NKp46 binder can subsequently be identified by comparison of samples subjected to trypsin digestion and samples incubated with antibody and then subjected to digestion by e.g. trypsin (thereby revealing a footprint for the binder). Other enzymes like chymotrypsin, pepsin, etc., also or alternatively can be used in similar epitope characterization methods. Moreover, enzymatic digestion can provide a quick method for analyzing whether a potential antigenic determinant sequence is within a region of the NKp46 polypeptide that is not surface exposed and, accordingly, most likely not relevant in terms of immunogenicity/antigenicity. See, e.g., Manca, Ann 1st Super Sanita. 1991; 27: 15-9 for a discussion of similar techniques.

Site-directed mutagenesis is another technique useful for elucidation of a binding epitope. For example, in “alanine-scanning”, each residue within a protein segment is replaced with an alanine residue, and the consequences for binding affinity measured. If the mutation leads to a significant reduction in binding affinity, it is most likely involved in binding. Monoclonal antibodies specific for structural epitopes (i.e., antibodies which do not bind the unfolded protein) can be used to verify that the alanine-replacement does not influence over-all fold of the protein. See, e.g., Clackson and Wells, Science 1995; 267:383-386; and Wells, Proc Natl Acad Sci USA 1996; 93:1-6.

Electron microscopy can also be used for epitope “foot-printing”. For example, Wang et al., Nature 1992; 355:275-278 used coordinated application of cryoelectron microscopy, three-dimensional image reconstruction, and X-ray crystallography to determine the physical footprint of a Fab-fragment on the capsid surface of native cowpea mosaic virus.

Other forms of “label-free” assay for epitope evaluation include surface plasmon resonance (SPR, BIACORE) and reflectometric interference spectroscopy (RifS). See, e.g., Fagerstam et al., Journal Of Molecular Recognition 1990; 3:208-14; Nice et al., J. Chromatogr. 1993; 646:159-168; Leipert et al., Angew. Chem. Int. Ed. 1998; 37:3308-3311; KrOger et al., Biosensors and Bioelectronics 2002; 17:937-944.

It should also be noted that an antibody binding the same or substantially the same epitope as an antibody of the invention can be identified in one or more of the exemplary competition assays described herein.

Once antibodies are identified that are capable of binding NKp46 and/or having other desired properties, they will also typically be assessed, using standard methods including those described herein, for their ability to bind to other polypeptides, including unrelated polypeptides. Ideally, the antibodies only bind with substantial affinity to NKp46, e.g., human NKp46, and do not bind at a significant level to unrelated polypeptides. However, it will be appreciated that, as long as the affinity for NKp46 is substantially greater (e.g., 5×, 10×, 50×, 100×, 500×, 1000×, 10,000×, or more) than it is for other, unrelated polypeptides), then the antibodies are suitable for use in the present methods.

The binding of the antibodies to NKp46-expressing cells can also be assessed in non-human primates, e.g. cynomolgus monkeys, or other mammals such as mice. The invention therefore provides an antibody, as well as fragments and derivatives thereof, wherein said antibody, fragment or derivative specifically bind NKp46, and which furthermore bind NKp46 from non-human primates, e.g., cynomolgus monkeys.

Upon immunization and production of antibodies in a vertebrate or cell, particular selection steps may be performed to isolate antibodies as claimed. In this regard, in a specific embodiment, the invention also relates to methods of producing such antibodies, comprising: (a) immunizing a non-human mammal with an immunogen comprising a NKp46 polypeptide; and (b) preparing antibodies from said immunized animal; and (c) selecting antibodies from step (b) that are capable of binding NKp46.

In one aspect of any of the embodiments, the antibodies prepared according to the present methods are monoclonal antibodies. In another aspect, the non-human animal used to produce antibodies according to the methods of the invention is a mammal, such as a rodent, bovine, porcine, fowl, horse, rabbit, goat, or sheep.

According to an alternate embodiment, the DNA encoding an antibody that binds an epitope present on NKp46 polypeptides is isolated from the hybridoma of this invention and placed in an appropriate expression vector for transfection into an appropriate host. The host is then used for the recombinant production of the antibody, or variants thereof, such as a humanized version of that monoclonal antibody, active fragments of the antibody, chimeric antibodies comprising the antigen recognition portion of the antibody, or versions comprising a detectable moiety.

DNA encoding the monoclonal antibodies of the invention, e.g., antibody Bab281, 9E2 or 195314, can be readily isolated and sequenced using conventional procedures (e.g., by using oligonucleotide probes that are capable of binding specifically to genes encoding the heavy and light chains of murine antibodies). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. As described elsewhere in the present specification, such DNA sequences can be modified for any of a large number of purposes, e.g., for humanizing antibodies, producing fragments or derivatives, or for modifying the sequence of the antibody, e.g., in the antigen binding site in order to optimize the binding specificity of the antibody.

Recombinant expression in bacteria of DNA encoding the antibody is well known in the art (see, for example, Skerra et al., Curr. Opinion in Immunol., 5, pp. 256 (1993); and Pluckthun, Immunol. 130, p. 151 (1992).

### Assessing the Ability of Antibodies to Modulate NKp46 Signaling

In certain embodiments, the antibodies of this invention are able to modulate, e.g., inhibit signaling by, NKp46 polypeptides, and consequently to modulate the activity and/or reactivity of NK cells during NK maturation. For example, antibodies may inhibit the activation of NKp46-expressing cells, e.g. they can inhibit the NKp46 signaling pathway, optionally with or without blocking the binding to NKp46 of natural or endogenous ligands; optionally they may block the ability of NKp46 protein to silence or down-modulate the silencing of the transcription factor Helios in the presence of a NKp46 ligand (e.g. in developing CD11b− NK cells or CD11b+ NK cells), thus modulating the activating state of NK cells during the NK cell maturation process. These antibodies are thus referred to as “neutralizing” or “inhibitory” or “blocking” antibodies. Such antibodies are useful, inter alia, for increasing the activity of NKp46-expressing immune cells, e.g. for the treatment or prevention of conditions where increasing NK cell activity can ameliorate, prevent, eliminate, or in any way improve the condition or any symptom thereof.

A range of cellular assays can be used to assess the ability of the antibodies to modulate NKp46 signaling. Any of a large number of assays, including molecular, cell-based, and animal-based models can be used to assess the ability of anti-NKp46 antibodies to modulate NKp46-expressing cell activity. Assays include, without limitation, any of the assays in the “Examples” section herein.

In one example, an anti-NKp46 antibody is tested and selected based on the ability of an anti-NKp46 antibody to “block” the binding of an NKp46 molecule and a natural ligand of an NKp46 receptor, in an assay using soluble or cell-surface associated NKp46 and an

NKp46 ligand. The antibody can preferably detectably reduce the binding of a NKp46 molecule to an NKp46 ligand in a dose-dependent fashion, where the NKp46 molecule detectably binds to the NKp46 ligand in the absence of the antibody. For example, NK cells can be brought into contact with cells expressing a ligand of NKp46, e.g. autologous CD11c+ spleen cells. The anti-NKp46 can be selected if it blocks activation of NK cells by the ligand expressing cells (the CD11c+ spleen cells).

In another example, a blocking NKp46 antibody neutralizes NKp46-mediated activation of NK cells (e.g. activation of NK cell cytotoxicity, CD107 expression, IFNγ production) in vitro in an assay wherein NK cells are brought into contact with target cells (e.g. target cells that are recognized and/or lysed by NK cells). While such antibody will inhibit NK cell activity (e.g. cytotoxicity) in an in vitro assay, when administered to mammal in vivo over a period sufficient to block NKp46 on developing NK cells the antibody will lead to NK cells with increased reactivity and/or activity. For example, an antibody can be selected for the ability to decrease specific lysis by NK cells by more than about 20%, preferably with at least about 30%, at least about 40%, at least about 50%, or more of the specific lysis obtained at the same effector:target cell ratio with NK cells or NK cell lines that are not inhibited by an anti-NKp46 antibody, as measured by a classical in vitro chromium release test of cytotoxicity. Examples of protocols for classical cytotoxicity assays are described, for example, in Pessino et al, J. Exp. Med, 1998, 188 (5): 953-960; Sivori et al, Eur J Immunol, 1999. 29:1656-1666; Brando et al, (2005) J. Leukoc. Biol. 78:359-371; E1-Sherbiny et al, (2007) Cancer Research 67(18):8444-9; and Nolte-'t Hoen et al, (2007) Blood 109:670-673).

In other examples, an anti-NKp46 antibody can be selected based on its ability to block or neutralize any NKp46-mediated modulation of NK cell activity and/or reactivity during or upon NK cell maturation, as assessed in vitro. For example, an antibody can be selected that inhibits NKp46-mediated silencing of Helios transcripts, and thereby lead to an increase in Helios transcripts or activity (e.g. in CD11b− NK cells or CD11b+ NK cells).

In one example, a reporter assay is used in which NKp46 ligand-expressing target cells are brought into contact with a NKp46 expressing cell (e.g. an NK cell, a T cell), and the ability of the antibody to block NKp46 signaling is assessed. For example, the target cell may be the DO.11.10 T cell hybridoma transduced with retroviral particles encoding a chimeric protein in which the intracytoplasmic domain of mouse CD3ζ is fused to the extracellular portion of NKp46 (DOMSP46), as described herein in Example 2. Engagement of the chimeric proteins at the cell surface triggers IL-2 secretion. After incubation, cell supernatants are assayed for the presence of mouse IL-2 in a standard target cell survival assay.

Additionally, or alternatively, an anti-NKp46 antibody can be selected based on its ability to block or neutralize NKp46-mediated modulation of NK cell activity and/or reactivity during or upon NK cell maturation in vivo. For example, a candidate NK cell antibody can be administered to a non-human mammal, optionally following depletion of NK cells, for a period of time sufficient (e.g. at least 10 or 11 days) to allow the emergence of NK cells whose NKp46 have been blocked during NK cell development and maturation. NK cells can be obtained from such mammal and tested for their reactivity and/or activity. For example, an antibody can be selected if it causes an increase in the reactivity or cytoxicity of NK cells toward target cells (infected cells, tumor cells, pro-inflammatory cells, etc.), increased activation, activation markers (e.g. CD107 expression) and/or IFNγ production in NK cells, and/or increased the frequency in vivo of such activated, reactive, cytotoxic and/or activated NK cells. In another example, NK cells can be obtained from such mammal and tested for Helios activity (e.g. detection and/or quantification of nucleic acid transcripts encoding Helios, the presence of a Helios polypeptide, or any biological activity mediated by Helios). An anti-NKp46 antibody of invention may be selected to inhibit NKp46-mediated silencing of Helios transcripts, and thereby lead to an increase in Helios transcripts or activity (e.g. in CD11b− NK cells or CD11b+ NK cells). In one example, the non-human mammal is a mammal that expresses a human NKp46 polypeptide on its NK cells, e.g. a NKp46 Tg mouse as described in PCT application publication WO2006/103569 (Innate Pharma and INSERM) and Walzer T., et al. (2007) Proc. Nat. Acad. Sci. USA 104(9):3384-3389, the disclosures of which are incorporated herein by reference.

Briefly, a non-human mammal that expresses a human NKp46 polypeptide on its NK cells method can be prepared in a method comprising:
a) providing an expression construct comprising an NKp46 promoter of a mammal, optionally a human NKp46 promoter, operably linked to a nucleic acid encoding said NKp46 polypeptide; and

b) introducing said construct into a nonhuman mammal, wherein said NKp46 polypeptide is expressed within NK cells of said nonhuman mammal. Preferably said NKp46 polypeptide is not expressed in any cell types other than NK cells within said nonhuman mammal.

In one embodiment, said protein is a mutated NKp46 polypeptide that affects the reactivity and/or activity of NK cells, optionally a NKp46 polypeptide having a mutation at position W32 (e.g. a W32R mutation), and said method is performed to produce an animal model for an increase of NK cell reactivity and/or activity. Optionally, the mutated NKp46 polypeptide decreases NKp46 activity and increases the reactivity and/or activity of NK cells, and said method is performed to produce an animal model for an increase of NK cell reactivity and/or activity. In one aspect, said method is performed to produce an animal model for disorders characterized by an increase of NK cell reactivity and/or activity. The invention also encompasses a nonhuman transgenic mammal produced using the method herein, as well as an NK cell isolated from any such nonhuman transgenic mammal.

In another embodiment, the invention provides a transgenic nonhuman mammal comprising a cell comprising a mammalian NKp46 promoter operably linked to a nucleic acid encoding a NKp46 polypeptide, wherein said nucleic acid comprises a mutation that results in lack of NKp46 expression or an decrease in NKp46 activity. Preferably the nucleic acid sequence encodes a mutated NKp46 polypeptide. Optionally, the NKp46 polypeptide is a heterologous (e.g. human) polypeptide.

In one embodiment, the invention provides a method for assessing the effect of a test compound on a nonhuman mammal, said method comprising:
a) providing a nonhuman transgenic mammal as described herein, or a nonhuman mammal to which has been administered a cell from a nonhuman transgenic mammal as described herein, said mammal expressing an NKp46 polypeptide (e.g. a heterologous NKp46 polypeptide, a human NKp46 polypeptide, a mutated NKp46 polypeptide) in an NK cell or expressing a mutated NKp46 nucleic acid in an NK cell;
b) administering to said mammal a test compound, optionally an anti-NKp46 antibody (e.g. an antibody that inhibits NKp46); and
c) assessing the effect of said test compound on said animal.

In one embodiment, the invention provides a method for assessing the effect of a test compound, said method comprising:
a) providing a nonhuman transgenic mammal comprising a cell comprising a mammalian (e.g. human) NKp46 promoter operably linked to a nucleic acid encoding a heterologous (e.g. human) NKp46 polypeptide;
b) administering to said mammal a test compound, optionally an anti-NKp46 antibody, that inhibits NKp46; and
c) assessing the effect of said test compound on said animal, optionally assessing the effect of said test compound on NK cell reactivity and/or activity.

With respect to any of the parameters tested in any of the in vitro or in vivo assays herein, an increase or decrease can be, e.g. an increase or decrease, respectively, of more than about 20%, preferably with at least about 30%, at least about 40%, at least about 50%, 60%, 80% or more, compared to that observed in the absence of treatment with anti-NKp46 antibody.

Helios (IKZF2) amino acid and nucleic acid sequences are well known in the art; for human sequences see e.g., NCBI accession number NM_{—}016260 (transcript variant 1 mRNA) and NCBI accession number NM_{—}001079526 (transcript variant 2 mRNA). Human Helios amino acid sequences are also provided in NP_{—}057344 (isoform 1):

(SEQ ID NO: 2)
METEAIDGYITCDNELSPEREHSNMAIDLTSSTPNGQHASPSHM
TSTNSVKLEMQSDEECDRKPLSREDEIRGHDEGSSLEEPLIESSEVADNRKVQELQGE
GGIRLPNGKLKCDVCGMVCIGPNVLMVHKRSHTGERPFHCNQCGASFTQKGNLLRHIK
LHSGEKPFKCPFCSYACRRRDALTGHLRTHSVGKPHKCNYCGRSYKQRSSLEEHKERC
HNYLQNVSMEAAGQVMSHHVPPMEDCKEQEPIMDNNISLVPFERPAVIEKLTGNMGKR
KSSTPQKFVGEKLMRFSYPDIHFDMNLTYEKEAELMQSHMMDQAINNAITYLGAEALH
PLMQHPPSTIAEVAPVISSAYSQVYHPNRIERPISRETADSHENNMDGPISLIRPKSR
PQEREASPSNSCLDSTDSESSHDDHQSYQGHPALNPKRKQSPAYMKEDVKALDTTKAP
KGSLKDIYKVFNGEGEQIRAFKCEHCRVLFLDHVMYTIHMGCHGYRDPLECNICGYRS
QDRYEFSSHIVRGEHTFH.

### CDRs, Fragments and Derivatives of Antibodies

Fragments and derivatives of antibodies of this invention (which are encompassed by the term “antibody” or “antibodies” as used in this application, unless otherwise stated or clearly contradicted by context), preferably a Bab281, 9E2 or 195314-like antibody, can be produced by techniques that are known in the art. “Fragments” comprise a portion of the intact antibody, generally the antigen binding site or variable region. Examples of antibody fragments include Fab, Fab′, Fab′-SH, F (ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments.

Fragments of the present antibodies can be obtained using standard methods. For instance, Fab or F (ab′) 2 fragments may be produced by protease digestion of the isolated antibodies, according to conventional techniques. It will be appreciated that immunoreactive fragments can be modified using known methods, for example to slow clearance in vivo and obtain a more desirable pharmacokinetic profile the fragment may be modified with polyethylene glycol (PEG). Methods for coupling and site-specifically conjugating PEG to a Fab′ fragment are described in, for example, Leong et al, 16 (3): 106-119 (2001) and Delgado et al, Br. J. Cancer 73 (2): 175-182 (1996), the disclosures of which are incorporated herein by reference.

Alternatively, the DNA of a hybridoma producing an antibody of the invention, preferably a Bab281, 9E2 or 195314-like antibody, may be modified so as to encode a fragment of the invention. The modified DNA is then inserted into an expression vector and used to transform or transfect an appropriate cell, which then expresses the desired fragment.

In certain embodiments, the DNA of a hybridoma producing an antibody of this invention, preferably a Bab281, 9E2 or 195314-like antibody, can be modified prior to insertion into an expression vector, for example, by substituting the coding sequence for human heavy- and light-chain constant domains in place of the homologous non-human sequences (e.g., Morrison et al., PNAS pp. 6851 (1984)), or by covalently joining to the immunoglobulin coding sequence all or part of the coding sequence for a non-immunoglobulin polypeptide. In that manner, “chimeric” or “hybrid” antibodies are prepared that have the binding specificity of the original antibody. Typically, such non-immunoglobulin polypeptides are substituted for the constant domains of an antibody of the invention.

Thus, according to another embodiment, the antibody of this invention, preferably a Bab281, 9E2 or 195314-like antibody, is humanized. “Humanized” forms of antibodies according to this invention are specific chimeric immunoglobulins, immunoglobulin chains or fragments thereof (such as Fv, Fab, Fab′, F (ab′)2, or other antigen-binding subsequences of antibodies) which contain minimal sequence derived from the murine immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody.

In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al., Nature, 321, pp. 522 (1986); Reichmann et al, Nature, 332, pp. 323 (1988); Presta, Curr. Op. Struct. Biol., 2, pp. 593 (1992); Verhoeyen et Science, 239, pp. 1534; and U.S. Pat. No. 4,816,567, the entire disclosures of which are herein incorporated by reference.) Methods for humanizing the antibodies of this invention are well known in the art.

The choice of human variable domains, both light and heavy, to be used in making the humanized antibodies is very important to reduce antigenicity. According to the so-called “best-fit” method, the sequence of the variable domain of an antibody of this invention is screened against the entire library of known human variable-domain sequences. The human sequence which is closest to that of the mouse is then accepted as the human framework (FR) for the humanized antibody (Sims et al., J. Immunol. 151, pp. 2296 (1993); Chothia and Lesk, J. Mol. 196, pp. 901). Another method uses a particular framework from the consensus sequence of all human antibodies of a particular subgroup of light or heavy chains. The same framework can be used for several different humanized antibodies (Carter et al., PNAS 89, pp. 4285 (1992); Presta et J. Immunol., 51, p. 1993)).

It is further important that antibodies be humanized with retention of high affinity for NKp46 receptors and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen (s), is achieved. In general, the CDR residues are directly and most substantially involved in influencing antigen binding.

Another method of making “humanized” monoclonal antibodies is to use a XenoMouse (Abgenix, Fremont, Calif.) as the mouse used for immunization. A XenoMouse is a murine host according to this invention that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference.

Human antibodies may also be produced according to various other techniques, such as by using, for immunization, other transgenic animals that have been engineered to express a human antibody repertoire (Jakobovitz et Nature 362 (1993) 255), or by selection of antibody repertoires using phage display methods. Such techniques are known to the skilled person and can be implemented starting from monoclonal antibodies as disclosed in the present application.

The antibodies of the present invention, preferably a Bab281, 9E2 or 195314-like antibody, may also be derivatized to “chimeric” antibodies (immunoglobulins) in which a portion of the heavy/light chain(s) is identical with or homologous to corresponding sequences in the original antibody, while the remainder of the chain (s) is identical with or homologous to corresponding sequences in antibodies derived from another species or belonging to another antibody class or subclass, as well as fragments of such antibodies, so long as they exhibit the desired biological activity and binding specificity (Cabilly et al., supra; Morrison et al., Proc. Natl. Acad. Sci. U.S.A., pp. 6851 (1984)).

Particularly preferred anti-NKp46 antibodies comprise an Fc portion of human IgG4 isotype. Such antibodies can be directly screened for such isotype or alternatively, the DNA of a hybridoma producing an antibody of the invention, preferably a Bab281, 9E2 or 195314-like antibody, may be modified so as to encode an Fc portion of human IgG4 isotype. The modified DNA is then inserted into an expression vector and used to transform or transfect an appropriate cell, which then expresses the desired fragment. In one preferred embodiment, the anti-NKp46 antibody comprises a heavy chain of human IgG4 isotype. Human IgG4 constant heavy chain region amino acid sequences are well known in the art: GenBank accession #: K01316 encodes a Human IgG4 constant heavy chain region polypeptide. In one embodiment, an anti-NKp46 antibody comprises an heavy chain comprising an amino acid sequence as shown in SEQ ID NO 3, a sequence at least 50%, 60%, 70%, 80%, 90%, 95% or 98% identical to SEQ ID NO 3 or a sequence at least 50%, 60%, 70%, 80%, 90%, 95% or 98% identical to a contiguous sequence of 20, 30, 50, 100 or 200 amino acid residues of SEQ ID NO 3.

(SEQ ID NO: 3)
STKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPSCPAPEFLGGPSVFLF
PPKPKDTLMISRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVDKSRWQEGNVFS
CSVMHEALHNHYTQKSLSLSLGK.

In one embodiment, the anti-NKp46 antibody comprises an IgG4 heavy chain comprising a serine to proline mutation in residue 241, corresponding to position 228 according to the EU-index (Kabat et al., “Sequences of proteins of immunological interest”, 5^{th} ed., NIH, Bethesda, M L, 1991). Compositions comprising such antibodies can be characterized as having less than about 15%, such as less than about 10% (e.g., about 5% or less, about 4% or less, about 3% or less, or even about 1% or less) of IgG4 “half-antibodies” (comprising a single heavy chain/light chain pair). Such IgG4 “half-antibody” by-products form due to heterogeneity of inter-heavy chain disulphide bridges in the hinge region in a proportion of secreted human IgG4 (see Angal et al., Molecular Immunology, 30(1):105-108, 1993 for a description of IgG4 “half-antibodies”, S241 P mutation, and related principles). This effect is typically only detectable under denaturing, non-reducing conditions.

Further, sites can be removed that affect binding to Fc receptors other than an FcRn salvage receptor in the antibodies of the invention. For example, the Fc receptor binding regions involved in ADCC activity can be removed in the antibodies of the invention. For example, mutation of Leu234/Leu235 in the hinge region of IgGI to L234A/L235A or Phe235/Leu236 in the hinge region of IgG4 to P235A/L236A minimizes FcR binding and reduces the ability of the immunoglobulin to mediate complement dependent cytotoxicity and ADCC. In one embodiment, the antibodies of the invention will comprise an IgG4 Fc domain with P235A/L236A mutations. The location of these residues identified above is typical in a mature heavy chain but can change depending on CDR lengths.

In one embodiment, the invention provides an antibody that binds essentially the same epitope or determinant as monoclonal antibody Bab281, 9E2 or 195314; optionally the antibody comprises an antigen binding region of antibody Bab281, 9E2 or 195314. In any of the embodiments herein, antibody Bab281, 9E2 or 195314 can be characterized by its amino acid sequence and/or nucleic acid sequence encoding it. In one preferred embodiment, the monoclonal antibody comprises the Fab or F(ab′)₂ portion of Bab281, 9E2 or 195314. Also provided is a monoclonal antibody that comprises the heavy chain variable region of Bab281, 9E2 or 195314. According to one embodiment, the monoclonal antibody comprises the three CDRs of the heavy chain variable region of Bab281, 9E2 or 195314. Also provided is a monoclonal antibody that further comprises the variable light chain variable region of Bab281, 9E2 or 195314 or one, two or three of the CDRs of the light chain variable region of Bab281, 9E2 or 195314. The sequences of the CDRs of the antibodies can be determined according to AbM (Oxford Molecular's AbM antibody modelling software definition), Kabat or Chothia definitions systems. Optionally any one or more of said light or heavy chain CDRs may contain one, two, three, four or five amino acid modifications (e.g. substitutions, insertions or deletions). Optionally, provided in an antibody where any of the light and/or heavy chain variable regions comprising part or all of an antigen binding region of antibody Bab281, 9E2 or 195314 are fused to an immunoglobulin constant region of the IgG type, optionally a human constant region, preferably an IgG4 isotype.

### Antibody Formulations

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The antibodies of this invention may be employed in a method of modulating, e.g inhibiting, the activity of NKp46-expressing cells in a patient. This method comprises the step of contacting said composition with said patient. Such method will be useful for both prophylaxis and therapeutic purposes.

For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques.

Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added.

Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Several monoclonal antibodies have been shown to be efficient in clinical situations, such as Rituxan™ (rituximab), Herceptin™ (Trastuzumab) or Xolair™ (Omalizumab), and similar administration regimens (i.e., formulations and/or doses and/or administration protocols) may be used with the antibodies of this invention. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of an anti-NKp46 antibody of the invention.

According to another embodiment, the antibody compositions of this invention may further comprise another therapeutic agent, including agents normally utilized for the particular therapeutic purpose for which the antibody is being administered. The additional therapeutic agent will normally be present in the composition in amounts typically used for that agent in a monotherapy for the particular disease or condition being treated. Such therapeutic agents include, but are not limited to anti-inflammation agents, steroids, immune system suppressors, antibiotics, antivirals and other antibodies and fragments thereof.

### Uses in Therapy

The antibodies and other compounds described herein can be used to prevent or treat disorders that benefit from enhanced NK cell activity, such as cancers, solid and non solid tumors, hematological malignancies, infections such as viral infections, and inflammatory or autoimmune disorders. The present invention also provides methods for identifying patients suitable for treatment using the present antibodies or compounds, or for identifying individuals suitable for inclusion in clinical trials designed to assess the therapeutic efficacy of the compounds of the present invention. In particular, individuals having diseases involving cells (target cells for elimination by NK cells) with elevated levels of ligands of NK cell activatory receptors on their surface are particularly well suited for such treatments or for inclusion in such a clinical trial.

As demonstrated herein, blocking anti-NKp46 antibodies when administered in vivo are particularly effective at increasing the activity of NK cells (or increasing the frequency of active NK cells). The antibodies inhibit NKp46, e.g., by blocking NKp46 signaling, thereby blocking or neutralizing NKp46-mediated modulation of NK cell reactivity and/or activity during maturation and as a result enhancing NK cell activity against target cells. The antibodies preferably comprise human heavy chain constant regions sequences but will not deplete NK cells to which they are bound and preferably do not comprise an Fc portion that induces ADCC. The composition further comprises a pharmaceutically acceptable carrier. Such compositions are also referred to as “antibody compositions” of the invention. In one embodiment, antibody compositions of this invention comprise an antibody disclosed in the antibody embodiments above.

The invention further provides a method of modulating NK cell activity in a patient in need thereof, comprising the step of administering to said patient a composition according to the invention. In one embodiment, NK cell activity is enhanced in a patient, wherein the patient has a disease or disorder wherein such NK cell activity may promote, enhance, and/or induce a therapeutic effect (or promotes, enhances, and/or induces such an effect in at least a substantial proportion of patients with the disease or disorder and substantially similar characteristics as the patient, as may determined by, e.g., clinical trials).

The invention also provides a method of enhancing NK cell activity in a patient in need thereof, comprising the step of administering a composition according to the invention to said patient. The method is more specifically directed at increasing NK cell activity in patients having a disease in which increased NK cell activity is beneficial, which involves, affects or is caused by cells susceptible to lysis by NK cells, or which is caused or characterized by insufficient NK cell activity, such as a cancer, another proliferative disorder, an infectious disease or an inflammatory or autoimmune disorder.

In one aspect, the methods of treatment of the invention comprise administering to an individual a composition comprising a compound that inhibits NKp46 (e.g. an anti-NKp46 antibody) in a therapeutically effective amount. A therapeutically effective amount may be for example an sufficient to cause an increase in the frequency of activated, reactive, cytotoxic and/or IFNγ-producing NK cells. Preferably the compound that inhibits NKp46 is administered at a dose and frequency that results in an inhibition of NKp46 over a sufficient amount of time to modulate NK cell maturation (e.g. inhibit NK cell maturation caused by NKp46-mediated silencing of the hellos transcription factor) and allow the emergence of a substantial increase in the frequency of activated, reactive (e.g. hyper-reactive), cytotoxic and/or IFNγ-producing NK cells. In one embodiment, the method results in an increase of at least 20%, 30%, 50%, 60%, 70%, 80%, 90% or 100% in the frequency of activated, reactive (e.g. hyper-reactive), cytotoxic and/or IFNγ-producing NK cells. In one embodiment, the method comprises repeating the administration at least once, for example with a dosing frequency in the range of 3 times per day to once per 2 months. The dose may also be administered, e.g., at least 3 times, at least 6 times, or at least 10 times.

In one embodiment, the dose is selected to provide full saturation (at least 90% occupancy of the NKp46 on NK cells) in human patients. The method optionally includes assessing the patient for NK cell activity or receptor saturation (which may be performed by use of any suitable technique, several of which being known in the art, including, e.g., NKp46 occupancy level, CD107a marker, IFNγ production, etc., as described herein). The formulation is typically administered by i.v. administration over a suitable period of time, such as about 1 hour.

For example, an anti-NKp46 antibody can be administered at a dose and a dosing frequency achieving at least about 50%, 60%, 70%, 80%, 90%, preferably at least about 95% NKp46 occupancy on NK cells in plasma for at least about one week, two weeks, one month, two months, three months or six months, thereby having sustained saturation for an extended period of time (e.g., at least 1, 2, 3 months, 6 months). The dosing frequency may be in the range of once per day to once per 2 months, from about once per week to about once per 2 months; or about once per month. Alternatively, the dosing frequency can be selected from about three times, about twice, and about once per day; about five times, about four times, about three times, and about twice per week; and about once every two, four, and six weeks.

In one preferred embodiment, a dose of anti-NKp46 antibody resulting in substantial receptor saturation (e.g. at least about 50%, 60%, 70%, 80%, 90%, receptor occupancy) is administered from about 2 times per week to about once per month, or from about once per month to about once per 2 months. The dose can be, e.g., administered at least 3 times, at least 6 times, or more. For example, the method may comprise administering an anti-NKp46 antibody at a dose and a dosing frequency achieving at least about 50%, 60%, 70%, 80%, 90%, NKp46 occupancy on NK cells for at least about two weeks, one month, 6 months, 9 months or 12 months.

In another aspect, the NKp46 antigen-binding compound is dosed in amount and at a frequency that results in substantial or substantially complete saturation of NKp46 on NK cells for a period of at least about 1 week, 2 weeks, 3 weeks or one month, and that permits a significant “de-saturation” during the treatment period prior to the subsequent administration of anti-NKp46 antibody. Since anti-NKp46 antibodies may inhibit the ability of NK cells to recognize and lyse target cells via their NKp46 polypeptides, such de-saturation may permit maximal activity of the hyper-reactive NK cells that have developed during the period in which the NK cells' NKp46 was blocked by the anti-NKp46 antibody. In one embodiment, a therapeutically active amount of one or more anti-NKp46 antibodies is an amount of such antibody that results in substantial or substantially complete NKp46 saturation on circulating NK cells for a period of at least about 1 week, 2 weeks, optionally about 3 weeks, optionally about one month, following administration of the antibody, and the antibody is dosed at least twice, following a period of de-saturation (e.g. 1, 2, 3, 4, 5 or 6 months). For example, dosing can occurs about (or at least) once every two, three, four, five or six months (subsequent doses are separated by about (or at least) two, three, four, five or six months).

Cancer

The methods of the present invention are utilized advantageously for the treatment of cancers and other proliferative diseases including, but not limited to, carcinoma, including that of the bladder, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma.

Other preferred disorders that can be treated according to the invention include hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sezary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukaemia (T-Lbly/T-ALL). Other proliferative disorders can also be treated according to the invention, including for example hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

Infectious Disease

The compositions according to the invention can be used to treat or prevent infectious diseases, including preferably any infections caused by viruses, bacteria, protozoa, molds or fungi. Such viral infectious organisms include, but are not limited to, hepatitis type A, hepatitis type B, hepatitis type C, influenza, varicella, adenovirus, herpes simplex type I (HSV-1), herpes simplex type 2 (HSV-2), rinderpest, rhinovirus, echovirus, rotavirus, respiratory syncytial virus, papilloma virus, papilloma virus, cytomegalovirus, echinovirus, arbovirus, huntavirus, coxsackie virus, mumps virus, measles virus, rubella virus, polio virus, Ebola-virus, and human immunodeficiency virus type I or type 2 (HIV-1, HIV-2).

Bacterial infections that can be treated according to this invention include, but are not limited to, infections caused by the following: Staphylococcus; Streptococcus, including S. pyogenes; Enterococcl; Bacillus, including Bacillus anthracis, and Lactobacillus; Listeria; Corynebacterium diphtheriae; Gardnerella including G. vaginalis; Nocardia; Streptomyces; Thermoactinomyces vulgaris; Treponema; Camplyobacter, Pseudomonas including Raeruginosa; Legionella; Neisseria including N. gonorrhoeae and N. meningitides; Flavobacterium including F. meningosepticum and F. odoraturn; Brucella; Bordetella including B. pertussis and B. bronchiseptica; Escherichia including E. coli, Klebsiella; Enterobacter, Serratia including S. marcescens and S. liquefaciens; Edwardsiella; Proteus including P. mirabilis and P. vulgaris; Streptobacillus; Rickettsiaceae including R. fickettsfi, Chlamydia including C. psittaci and C. trachomatis; Mycobacterium including M. tuberculosis, M. intracellulare, M. folluiturn, M. laprae, M. avium, M. bovis, M. africanum, M. kansasii, M. intracellulare, and M. lepraernurium; and Nocardia.

Protozoa infections that may be treated according to this invention include, but are not limited to, infections caused by leishmania, kokzidioa, and trypanosoma. A complete list of infectious diseases can be found on the website of the National Center for Infectious Disease (NCID) at the Center for Disease Control (CDC) (World Wide Web (www) at cdc.gov/ncidod/diseases/), which list is incorporated herein by reference. All of said diseases are candidates for treatment using the compositions according to the invention.

Autoimmune and Inflammatory Disorders

Diseases and conditions in which the present compounds that inhibit NKp46 can be used also include any diseases mediated or exacerbated partially or totally by T cells (e.g. CD4+ T cells, CD8+ T cells), including inter alia disorders such as inflammatory diseases and autoimmune diseases. The compounds that inhibit NKp46 can also be used to treat a patient undergoing transplantation, e.g. to prevent graft-versus-host disease.

In one embodiment, the compounds that inhibit NKp46 are used to treat an individual having an autoimmune or inflammatory disease that has is established, has signs of ongoing or active inflammation, has physical signs of disease (e.g. joint swelling, lesions, neurological symptoms, etc.), has chronic disease, has severe disease (as assessed by applicable criteria, e.g. DAS or ACR criteria in rheumatoid arthritis) or has progressing disease. “Established disease” refers to an autoimmune or inflammatory disease which has been declared for an extended period of time, e.g. more than one year. Depending on the specific disease, established disease also means a disease which is not controlled e.g. which is still progressing or for which the patient does not experience remission, in the presence or in the absence of a treatment. Compounds that inhibit NKp46 can also advantageously be used to treat chronic disease. “Chronic disease” refers to a disease that persists for an extended period of time. For instance, a chronic disease can be a disease lasting 3 months or more, as defined by the U.S. National Center for Health Statistics.

The methods of the present invention are utilized for the treatment of autoimmunity, inflammation, allergy, asthma, infections (e.g. chronic infection, viral infection) and sepsis. Examples of diseases which can be treated with the compounds that inhibit NKp46 include, but are not limited to arthritis, systemic lupus erythematosus, sepsis, asthma, osteoporosis, autoimmunity to central nervous system antigens, autoimmune diabetes, inflammatory bowel disease, autoimmune carditis, autoimmune hepatitis. Other immune disorders treatable using the compounds that inhibit NKp46 signaling according to the invention include, inter alia, autoimmune disorders and inflammatory disorders, including, but not limited to, Crohn's disease, Celiac disease, ulcerative colitis, irritable bowel syndrome, acute disseminated encephalomyelitis (ADEM), Addison's disease, antiphospholipid antibody syndrome (APS), aplastic anemia, autoimmune hepatitis, Diabetes mellitus, Goodpasture's syndrome, Graves' disease, Guillain-Barré syndrome (GBS), Hashimoto's disease, lupus erythematosus, demyelinating conditions, Multiple sclerosis, Myasthenia gravis, opsoclonus myoclonus syndrome (OMS), optic neuritis, Ord's thyroiditis, pemphigus, cirrhosis, psoriasis, rheumatoid arthritis, Reiter's syndrome, Takayasu's arteritis, temporal arteritis, warm autoimmune hemolytic anemia, Wegener's granulomatosis, appendicitis, arteritis, arthritis, blepharitis, bronchiolitis, bronchitis, bursitis, cervicitis, cholangitis, cholecystitis, chorioamnionitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gingivitis, hepatitis, hidradenitis suppurativa, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonitis, proctitis, prostatitis, pyelonephritis, rhinitis, salpingitis, sinusitis, stomatitis, synovitis, tendonitis, tonsillitis, uveitis, vaginitis, vasculitis, and vulvitis.

### Drug Combinations

In other embodiments, the method may comprise the additional step of administering to said patient an appropriate additional therapeutic agent useful in treatment or prevention of the disease from which the patient suffers or is susceptible to; examples of such agents include a chemotherapeutic agent, an immunomodulatory agent, a hormonal agent, an anti-inflammation drug, a steroid, an immune system suppressor, a corticosteroid, an antibiotic, an anti-viral or an adjunct compound. Such additional agents can be administered to a patient as a single dosage form together with said antibody, or as a separate dosage form. The dosage of the antibody (or antibody and the dosage of the additional therapeutic agent collectively) are sufficient to detectably induce, promote, and/or enhance a therapeutic response in the patient. Where administered separately, the antibody, fragment, or derivative and the additional therapeutic agent are desirably administered under conditions (e.g., with respect to timing, number of doses, etc.) that result in a detectable combined therapeutic benefit to the patient.

When cancer is being treated using the present anti-NKp46 antibodies, in another embodiment the method of the present invention comprises the additional step of administering to said patient another anti-cancer compound or subjecting the patient to another therapeutic approach. For solid tumor treatment, for example, the administration of a composition of the present invention may be used in combination with classical approaches, such as surgery, radiotherapy, chemotherapy, and the like. The invention therefore provides combined therapies in which the present oligonucleotides are used simultaneously with, before, or after surgery or radiation treatment; or are administered to patients with, before, or after conventional chemotherapeutic, radiotherapeutic or anti-angiogenic agents, or targeted immunotoxins or coaguligands.

Examplary anti-cancer anti-angiogenic agents inhibit signaling by a receptor tyrosine kinase including but not limited to FGFR (fibroblast growth factor receptor, FGF-1,2), PDGFR (platelet derived growth factor receptor), angiopoïetins receptors (Ang-1,2), HGFR (hepatocytary growth factor receptor), ephrines receptor (Eph), VEGFR1, VEGFR-2,3 PDGFR-α, PDGFR-β, CSF-1R, MET, Flt-3, c-Kit, bcr/abl, p38 alpha and FGFR-1. Further anti-angiogenic agents may include agents that inhibit one or more of the various regulators of VEGF expression and production, such as EGFR, flt-1, KDR HER-2, COX-2, or HIF-1α. Another preferred class of agents includes IMiD (immunomodulatory drugs), analogs derived from thalidomide that have a wide range of effects, including both immune and non-immune related effects. Representatives of the IMiD class include CC-5013 (lenalidomide, Revlimid™), CC-4047 (Actimid™), and ENMD-0995. Another class of anti-angiogenic agent includes cilengitide (EMD 121974, integrin inhibitor), metalloproteinases (MPP) such as marinastat (BB-251). Another class of anti-angiogenic agents includes farnesylation inhibitors such as lonafarnib (Sarasar™), tipifarnib (Zarnestra™). Other anti-angiogenic agents can also be suitable such as Bevacuzimab (mAb, inhibiting VEGF-A, Genentech); IMC-1121B (mAb, inhibiting VEGFR-2, ImClone Systems); CDP-791 (Pegylated DiFab, VEGFR-2, Celltech); 2C3 (mAb, VEGF-A, Peregrine Pharmaceuticals); VEGF-trap (Soluble hybrid receptor VEGF-A, P1GF (placenta growth factor) Aventis/Regeneron). Another preferred class of agents includes the tyrosine kinase inhibitor (TKI) class, including, e.g., PTK-787 (TKI, VEGFR-1,-2, Vatalanib, Novartis); AEE788 (TKI, VEGFR-2 and EGFR, Novartis); ZD6474 (TKI, VEGFR-1,-2,-3, EGFR, Zactima, AstraZeneca); AZD2171 (TKI, VEGFR-1,-2, AstraZeneca); SU11248 (TKI, VEGFR-1,-2, PDGFR, Sunitinib, Pfizer); AG13925 (TKI, VEGFR-1,-2, Pfizer); AG013736 (TKI, VEGFR-1,-2, Pfizer); CEP-7055 (TKI, VEGFR-1,-2,-3, Cephalon); CP-547,632 (TKI, VEGFR-1,-2, Pfizer); GW786024 (TKI, VEGFR-1,-2,-3, GlaxoSmithKline); GW786034 (TKI, VEGFR-1,-2,-3, GlaxoSmithKline); sorafenib (TKI, Bay 43-9006, VEGFR-1,-2, PDGFR Bayer/Onyx); SU4312 (TKI, VEGFR, PDGFR, Pfizer), AMG706 (TKI, VEGFR-1,-2,-3, Amgen), XL647 (TKI, EGFR, HER2, VEGFR, ErbB4, Exelixis), XL999 (TKI, FGFR, VEGFR, PDGFR, Flt-3, Exelixis), PKC412 (TKI, KIT, PDGFR, PKC, FLT3, VEGFR-2, Novartis), AEE788 (TKI, EGFR, HER2, VEGFR, Novartis), OSI-930 (TKI, c-kit, VEGFR, OSI Pharmaceuticals), OSI-817 (TKI, c-kit, VEGFR, OSI Pharmaceuticals), DMPQ (TKI, ERGF, PDGFR, erbB2, p56, pkA, pkC), MLN518 (TKI, FLT3, PDGFR, c-KIT, CT53518, Millennium Pharmaceuticals), lestaurinib (TKI, FLT3, CEP-701, Cephalon), ZD1839 (TKI, EGFR, gefitinib, Iressa, AstraZeneca), OSI-774 (TKI, EGFR, Erlotininb, Tarceva, OSI Pharmaceuticals), lapatinib (TKI, ErbB-2, EGFR, GD-2016, Tykerb, GlaxoSmithKline). Most preferred are tyrosine kinase inhibitors that inhibit one or more receptor tyrosine kinases selected from the group consisting of VEGFR-1, VEGFR-2, VEGFR-3, PDGFR-α, β, Flt-3, c-Kit, p38 alpha, MET, c-RAF, b-RAF, bcr/abl and FGFR-1.

In one embodiment, the second agent is a natural ligand of an NK cell activating or an antibody that binds and activates an NK cell activating receptor other than NKp46. In one embodiment the agent is an agent that increases the presence of a natural ligand of an NK cell activating receptor on the surface of an target cell (e.g., infected cells, tumor cells, pro-inflammatory cells). NK cell activating receptors include, for example, NKG2D or activating KIR receptors (KIR2DS receptors, KIR2DS2, KIR2DS4). As used herein, the term “activating NK receptor” refers to any molecule on the surface of NK cells that, when stimulated, causes a measurable increase in any property or activity known in the art as associated with NK activity, such as cytokine (for example IFN-γ and TNF-α) production, increases in intracellular free calcium levels, the ability to target cells in a redirected killing assay as described, e.g. elsewhere in the present specification, or the ability to stimulate NK cell proliferation. The term “activating NK receptor” includes but is not limited to activating forms or KIR proteins (for example KIR2DS proteins), NKG2D, IL-2R, IL-12R, IL-15R, IL-18R and IL-21R.

In one embodiment, the anti-cancer agent is a chemotherapeutic agents or radiation that upregulate expression of NKG2D ligands on the surface of tumor cells. These include well known chemotherapies including ionizing and UV radiation, inhibitors of DNA replication, inhibitors of DNA polymerase, chromatin modifying treatments, as well as apoptosis inducing agents such as HDAC inhibitors trichostatin A and valproic acid. Preferred therapies are those that activate the DNA damage response pathway, more preferably those that activate the ATM (ataxia telangiectasia, mutated) or ATR (ATM- and Rad3-related) protein kinases, or CHK1, or yet further CHK2 or p53. Examples of the latter include ionizing radiation, inhibitors of DNA replication, DNA polymerase inhibitors and chromatic modifying agents or treatment including HDAC inhibitors. Compositions that upregulate NKG2D ligands are further described in Gasser et al (2005) Nature 436(7054):1186-90. NKG2D is an activating receptor that interacts with the MHC class I-related MICA and MICB glycoproteins, among other ligands. MICA and MICB (Bauer et al. (1999) Science 285:727-729, the disclosure of which is incorporated herein by reference) have no role in antigen presentation, are generally only found in intestinal epithelium, and can be stress-induced in permissive types of cells by viral and bacterial infections, malignant transformation, and proliferation. NKG2D is a C-type lectin-like activating receptor that signals through the associated DAP10 adaptor protein, which is similar to CD28. It is expressed on most natural killer (NK) cells, NKT cells, γδ T cells CD8 T cells, and T cells, but not, in general, on CD4 T cells. Ligand engagement of NKG2D activates NK cells and potently co-stimulates effector T cells, however certain NKG2D ligands also induce potent inhibition of proliferation (Kriegeskorte et al. (2005) PNAS 102(33): 11805-11810). Expression of NKG2D in NK cells is controlled by ligand-induced down-modulation, which is transient and rapidly reversed in the presence of IL-15. Other NKG2D ligands include ULBP proteins, e.g., ULBP-1, -2, and -3, originally identified as ligands for the human cytomegalovirus glycoprotein UL16 (Cosman et al, (2001) Immunity 14: 123-133, the disclosure of which is incorporated herein by reference). These proteins are distantly related to MHC class I proteins, but they possess only the a1 and a2 Ig-like domains, and they have no capacity to bind peptide or interact with b2-microglobulin. Further NKG2D ligands include RAE1TG, a member of the ULBP-like family of proteins (Bacon et al (2004) J. Immunol. 173:1078-1084) and Letal (PCT patent publication no. WO 2004/022706, both of the foregoing disclosure incorporated herein by reference.

Further anti-cancer agents include alkylating agents, cytotoxic antibiotics such as topoisomerase I inhibitors, topoisomerase II inhibitors, plant derivatives, RNA/DNA antimetabolites, and antimitotic agents. Preferred examples may include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, taxol, gemcitabine, navelbine, transplatinum, 5-fluorouracil, vincristin, vinblastin and methotrexate, or any analog or derivative variant of the foregoing.

Alkylating agents are substances that form compounds that are highly chemically reactive and rapidly form covalent bonds with suitable substances. One such target is DNA, not in its normal state but when the double helix has been unpaired by helicases. This exposes the ‘inside’ of the DNA, which is susceptible to alkylation. Most alkylating agents are bipolar, i.e., they contain two groups capable of reacting with DNA. They can thus form ‘bridges’ between two parts of a single strand of DNA or two separate strands; either way, this interferes with the actions of the enzymes involved with the replication process, which are unable to complete their effects. The cell then either dies because it is physically unable to divide or because the abnormal DNA stimulates apoptosis. Examples include nitrogen mustards (e.g. chlorambucil, cyclophosphamide), nitrosureas (e.g. carmustine, lomustine), metal salts (e.g. cisplatin, carboplatin, oxaliplatin), ethylenamine derivatives (e.g. thiotepa), alkyl sulphonates (e.g. busulphan) and triazenes (e.g. dacarbazine).

Antimetabolites are a group of chemicals that are similar in structure or function to naturally occurring metabolites required for the synthesis of nucleic acids. Antimetabolite molecules mimic these normal metabolites and either block the enzymes responsible for nucleic acid synthesis or become incorporated into DNA, which produces an incorrect genetic code and leads to apoptosis. There are three main classes of antimetabolites. Folate is a substance that is necessary for the synthesis of purine molecules. Folate analogues (e.g. methotrexate, raltritrexed) are similar to the folate molecule—substances such as methotrexate can be used to inhibit the enzyme dihydrofolate reductase, resulting in insufficient production of the purine thymine. Pyrimidine analogues (e.g. cytarabine, fluoroacil (5-FU), gemcitabine) resemble pyrimidine molecules and work by either inhibiting the synthesis of nucleic acids (e.g. fluorouracil) or by becoming incorporated into DNA (e.g. cytarabine). Purine analogues (e.g. mercaptopurine, thioguanine, cladribine, fludarabine) work in similar ways to pyrimidine analogues, but may have additional (and ill-characterized) mechanisms of action.

Cytotoxic antibiotics are so called because they are all derived from a natural source, the Streptomyces group of bacteria. They affect the function and synthesis of nucleic acids in different ways. The anthracycline group includes doxorubicin, daunorubicin and idarubicin. They intercalate with DNA and affect the topoisomerase II enzyme. This DNA gyrase splits the DNA double helix and reconnects it once torsional forces have been relieved; the anthracyclines stabilize the DNA-topoisomerase II complex and thus prevent reconnection of the strands. Dactinomycin and mitoxantrone have a similar mechanism of action. Bleomycin causes fragmentation of DNA chains. Mitomycin functions similar to the alkylating agents, causing DNA cross-linkage.

Plant derivatives include the vinca alkaloids such as vincristine and vinblastine bind to precursors of microtubules, preventing their formation. This inhibits the process of mitosis. The taxanes (paclitaxel and docetaxel) also act on microtubules. They stabilize them in their polymerized state, which also causes the arrest of mitosis. Podophyllyum derivatives such as etoposide and teniposide are thought to inhibit topoisomerase II, while irinotecan and topotecan inhibit topoisomerase I.

When infectious diseases are treated, the treatment may employ a composition according to the invention, either alone or in combination with other treatments and/or therapeutic agents known for treating such diseases, including anti-viral agents, anti-fungal agents, antibacterial agents, antibiotics, anti-parasitic agents and anti-protozoal agents. When these methods involve additional treatments with additional therapeutic agents, those agents may be administered together with the antibodies of this invention as either a single dosage form or as separate, multiple dosage forms. When administered as a separate dosage form, the additional agent may be administered prior to, simultaneously with, of following administration of the antibody of this invention.

When inflammatory or autoimmune diseases are treated with a compound that inhibits NKp46, the treatment methods this invention may further comprise treating an individual with a second therapeutic agent, including agents normally utilized for the particular therapeutic purpose for which the antibody is being administered. The second therapeutic agent will normally be administered in amounts typically used for that agent in a monotherapy for the particular disease or condition being treated. In one embodiment, the second therapeutic agent is administered in a dose less than the generally accepted efficacious dose; for example, in various embodiments, the composition comprises a dosage that is less than about 10% to 75% of the generally accepted efficacious dose is administered. Preferably, the second therapeutic agent is an agent that reduces proteolytic enzymes, an inflammatory mediator, or a proinflammatory cytokine such as TNF-α and/or interleukin-1 (IL-1). Preferably, the second therapeutic agent is DMARD or a DMD, optionally further wherein the second therapeutic agent is methotrexate (Rheumatrex™, Trexall™), hydroxychloroquine (Plaquenil™), sulfasalazine (Azulfidine®), leflunomide (Arava™), a tumor necrosis factor inhibitor (e.g. a soluble TNFα receptor such as etanercept (Enbrel®), a neutralizing (preferably non-depleting) anti-TNFα antibody such as adalimumab (Humira™) or Certolizumab pegol (Cimzia™)), a T-cell costimulatory blocking agent (e.g. abatacept (Orencia™)), an interleukin-1 (IL-1) receptor antagonist therapy (anakinra (Kineret™)), an anti-BIyS antibody (Benlysta™), a proteosome inhibitor (e.g. bortezomib), a tyrosine kinase inhibitor, intramuscular gold, or another immunomodulatory or cytotoxic agent (e.g. azathioprine (Imuran™), cyclophosphamide, cyclosporine A (Neoral™, Sandimmune™)) or a kinase inhibitor (e.g. a SYK kinase inhibitor such as fostimatinib (R788) or a JAK1, JAK2 inhibitors such as INCB28050, tanezumab or tasocitinib (CP-690,550)).

In the treatment methods of the invention, the compound that inhibits NKp46 and the second therapeutic agent can be administered separately, together or sequentially, or in a cocktail. In some embodiments, the compound that inhibits NKp46 is administered prior to the administration of the second therapeutic agent. For example, a compound that inhibits NKp46 can be administered approximately 0 to 30 days prior to the administration of the second therapeutic agent. In some embodiments, a compound that inhibits NKp46 is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days prior to the administration of the second therapeutic agent. In some embodiments, a compound that inhibits NKp46 is administered concurrently with the administration of the therapeutic agents. In some embodiments, a compound that inhibits NKp46 is administered after the administration of the second therapeutic agent. For example, a compound that inhibits NKp46 can be administered approximately 0 to 30 days after the administration of the second therapeutic agent. In some embodiments, a compound that inhibits NKp46 is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days after the administration of the second therapeutic agent.

The present compounds that inhibit NKp46 can be included in kits. The kits may optionally further contain any number of antibodies and/or other compounds, e.g., 1, 2, 3, 4, or any other number of anti-NKp46 antibodies and/or other compounds. It will be appreciated that this description of the contents of the kits is not limiting in any way. For example, the kit may contain other types of therapeutic compounds. Preferably, the kits also include instructions for using the antibodies, e.g., detailing the herein-described methods.

Further aspects and advantages of this invention will be disclosed in the following experimental section, which should be regarded as illustrative and not limiting the scope of this application.

### EXAMPLES

### Example 1

### NKp46 Blockade Leads to Hyper-Reactive NK Cells and Diminished T Cell Responses

### Materials and Methods

1. Mice and ENU Mutagenesis

ENU-mutagenesis was performed on a C57BL/6J (Charles River) background as previously described (33). C57BL/6J CD45.1 were purchased from Charles River. C57BL/6J, Noé and huNKp46 Tg (15) littermates and NKDTR/eGFP mice (15) were bred and maintained under specific pathogen-free (SPF) conditions. Experiments were conducted in accordance with institutional guidelines for animal care and use.

2. MCMV Infection and Viral Titration

To study mouse resistance, mice were treated or not with 100 μg of anti-NK1.1 mAB (PK136) and infected intraperitoneally the day after with 1,600 to 7,500 PFU per gram of body weight of MCMV K181 strain. To study T cell responses, mice were infected with 1,600 PFU per gram of body. Measurement of viral titer in spleens and livers was performed after serial dilutions of organ homogenates in DMEM, 3% FCS that were incubated on 3T3-NIH cell overlays for 2 hours to allow virus attachment. Cells were covered with pre-warmed carboxymethylcellulose DMEM medium, cultured for 5 more days, then fixed with formalin, and colored with crystal violet.

Lm-OVA Infection and Bacterial Titration

An Lm strain that had been engineered to express OVA (Lm-OVA) was prepared from clones grown from the organs of infected mice. For mouse infections, bacteria were grown to the exponential growth phase (0D₆₀₀=0.05-0.15) in brain heart infusion (BHI) medium (Sigma-Aldrich), diluted in PBS and injected i.v. into the retroorbital vein. In all experiments, mice were subjected to primary immunization with a dose of 0.1×LD₅₀ of bacteria (1×10⁴). Secondary infections were performed 1 month later, with 5×LD₅₀ of Lm-OVA bacteria (5×10⁵). For the determination of bacterial titers in the spleen, organs were harvested and dissociated on nylon screens, in 10 ml of 0.1% Triton X-100 (Sigma-Aldrich). Serial dilutions were performed in the same buffer, and 100 μl was plated on BHI agar.

3. Anti-NKp46 In Vivo Treatment

NDE mice were treated with DT (Calbiochem) to allow NK cell depletion. Upon NK cell repopulation, mice were treated with 100 μg of anti-NKp46 (29A1.4) or rat IgG2a control antibody (eBiosciences) every 2-3 days for 11 days. On day 15, bone marrow and splenic NK cells were analyzed.

4. Antibodies

Monoclonal antibodies used for flow cytometry were: purified anti-NKp46 (29A1.4), -Alexa 647, PE, anti-NK1.1 (PK136)-APC, PerCP-Cy5.5 and purified, anti-CD3 (145-2C11)-PE, FITC, PerCP-Cy5.5 and APC, anti-CD11b (M1/70)-V450, anti-Ly49H (3D10)-Alexa 647, anti-CD8 (53-6.7)-PerCP-Cy5.5, anti-CD4(RM4-5)-pacific blue and APC, anti-CD45.1 (A20)-Pacific Blue, anti-IFN-γ (XMG1.2)-APC and Alexa 647, anti-CD107a (1D4B)-FITC and PE, anti-GM-CSF (MP1-22E9)-PE. All antibodies were purchased from BD Pharmingen. Anti-CD45.2 (104)-Alexa700 was purchased from eBiosciences. Anti-rat alexa-647 was purchased from invitrogen. Goat polyclonal sera anti-mouse NKp46, normal goat sera and anti-TGF-β (1D11) were purchased from R&D Systems and revealed by a donkey anti-goat Alexa 647 from Invitrogen. Human-NKp46 antibody (BAB281) was purchased from Beckman. H2D^{b}/m45₅₀₇₋₅₁₅-APC pentamers and m45₅₀₇₋₅₁₅ peptide were purchased from Prolmmune. Samples were analyzed using a FACSCanto II (BD Biosciences) and the FlowJo software (Three Star, Stanford, USA).

5. Cell Suspensions

Bone marrow cells were obtained by flushing femurs. Spleens were smashed on nylon screen in complete RPMI 1640 medium supplemented with 10% FCS. Red blood cells were lysed for 2-3 min in ACK buffer. Liver were cut in small pieces and incubated at 37° C. for 20 min in HBSS medium (Invitrogen) containing 4,000 U/ml collagenase I (Invitrogen). Lymphocytes were then enriched by Percoll gradient centrifugation (Amersham-Pharmacia).

6. Antibody Staining and Flow Cytometry

Fc receptors were blocked by incubation with anti-FcgRII/III (2.4G2) during 10 minutes on ice. Cells were then stained with the specified antibodies in 50 μl of PBS containing 2% FCS (FACS buffer). For intracellular staining, cells were surface stained, fixed in 1% paraformaldehyde FACS buffer for 10 min on ice, and further permeabilized for 30 min in 1×Perm/Wash (BD Biosciences) containing anti-IFN-γ. Cells were then washed in 1×Perm/Wash and resuspended in FACS buffer.

7. NK Cell Effector Activities During MCMV and Lm-OVA Infection

Spleen cells were put in culture in complete RPMI 1640 supplemented with 10% FCS for 4 hours in the presence of brefeldin (Golgi-Plug; BD Biosciences) and monensin (Golgi-Stop; BD Biosciences) and anti-CD107a antibody (1D4B, eBiosciences). Cells were then surface stained and intracellular IFN-γ was revealed.

8. NK Cell Stimulation

Spleen cell suspensions were put in culture in complete RPMI 1640 supplemented with 10% FCS and with 1000 U/ml of rIL-2 (Proleukin-Chiron) for 5 days. IL-2-activated NK cells were then incubated with YAC-1 tumor targets for 5 hours in the presence of monensin (Golgi-Stop; BD Biosciences) and anti-CD107a antibody (1D4B, eBiosciences). Cells were then surface stained and intracellular IFN-γ revealed.

For IL-12/IL-18 stimulation, spleen cells were incubated with 5 ng/ml IL-18 (R&D Systems) and various amounts of IL-12 (R&D Systems) for 5 hours in the presence of monensin and brefeldin. Cells were surface-stained and intracellular IFN-γ was revealed.

In some cases, spleen cell suspensions were distributed in a 96-well 2HB Immulon plate precoated with 25 μg/ml or indicated concentration of purified anti-NK1.1 antibody (PK136; eBiosciences) for 4 hours. When indicated, wortmannin (Sigma-Aldrich) was added to the culture. Cells were surface stained and intracellular IFN-γ was revealed.

For NK-CD11c⁺ cell co-culture, CD11c⁺ cells were enriched from spleen through incubation with CD11c microbeads (Miltenyi Biotec), according to the manufacturer's protocol. NK cells were obtained by negative enrichment from spleen by staining cells with rat anti-CD5, -CD4, -CD8, —IA/IE, -Ter119 antibodies and incubating with anti-rat antibody-coated magnetic beads. After negative enrichment, cells were stained with 1 μM CFSE (Molecular Probes) and co-culture with CD11c⁺-enriched splenocytes in a 1:1 ratio, for 5 hours in the presence or absence of anti-NKp46 or isotype control Fab′(2) mAbs plus brefeldin and monensin. Intracellular cytokines were then revealed.

9. T Cell Stimulation

Spleen cell suspensions were put in culture in complete RPMI 1640 supplemented with 10% FCS in the presence of 10 μM of H2D^{b}-restricted m45₅₀₇₋₅₁₅ peptide (Prolmmune) plus brefeldin and monensin. Liver suspensions were distributed in a 96-well plate coated with 15 μg/ml of anti-CD3 antibody in the presence of brefeldin and monensin. After 6 hours of culture, cells were surface stained and intracellular IFN-γ was revealed.

10. Generation of Mixed Bone Marrow Chimera

Recipient mice (C57BL/6-CD45.1, 9-week-old males, Charles river) were conditioned by 2×400 rad of lethal irradiation. Donor bone marrow cells were obtained from femurs and tibias of 9-week-old male C57BL/6J (CD45.2 or CD45.1) or Noé (CD45.2) mice and CD45.1+ and CD45.2⁺ cells were mixed at a 1:1 ratio. Donor cells (3.10⁶/mouse) were injected i.v. (retro-orbital) in recipient mice the day after irradiation. Mixed bone marrow chimeras were kept on antibiotic-containing water (0.28% pediatric suspension of Bactrim; Roche, Basel, Switzerland) for 3 weeks after injection. Mice were analyzed 8 weeks later.

11. Whole Genome Sequencing

Standard Illumina protocols were followed for creation of short-insert paired-end libraries (insert length ˜500 bp). Sequence reads of 2×114 bp reads were produced using Illumina GA2X sequencers following standard Illumina protocols. The sequence reads were mapped to the version 9 of the mouse genome with GEM (http://gemlibrary.sourceforge.net) allowing up to 4 mismatches. Only reads mapping once to the genome were retained for subsequent analyses in order to avoid spurious variant calls at repetitive regions. This pre-filtered GEM output was transformed to the SAM format (Li et al. 2009) by using the gem-2-sam function.

Variants were called in individual samples by using the pileup function with -vc options in samtools v0.1.9 (http://samtools.sourceforge.net/; Li et al. 2009). The raw variant calls were subsequently filtered by using the samtools.pl script provided in the same software suit with 5 and 80 as minimum and maximum read depth values, respectively.

Additional post-filters were applied to retain SNPs with SNP quality>20 and indel calls supported by more than one read event and with SNP quality>50. All retained variants were annotated by using an in-house modified version of the snp_effect_predictor.pl script based on the Ensembl API (http://www.ensembl.org) in a local mirror of the Ensembl 59 mouse database. The variants candidates to be ENU-induced mutations were highlighted with an in-house developed approach working on the top of the mpileup function of samtools. At each genome position covered at least 5 times in both samples, a Fisher exact test was performed comparing the reads supporting the reference or an alternative event in both samples. A variant was considered to be ENU-induced when the Fisher-exact test between the allele counts in the ENU and WT samples gave a p-value<0.0001, the alternative allele proportion was <0.1 in wild-type and >0.2 in ENU-mice and the variant passed the individual sample filters described above.

12. NKp46^{WN32R} Modeling

The structure of D1-D2 ectodomain of mouse NKp46 was mutated at the residue Tryptophane 32 (crystal structure numerotation) in Arginine using the FoldX plugin of Yasara homology modeling program (34). Molecular dynamics and stereochemistry validation were performed further using the FoldX default force fields parameters (35).

13. Helios Transcripts Analysis

Spleen and bone marrow cell suspensions from 3 mice per group were stained with rat anti-CD3, -CD5, -CD4 and -CD8 antibodies and incubated with anti-rat coated magnetic beads. After negative enrichment cells were stained with anti-NK1.1, -CD3, -CD11b antibodies. 60 to 90,000 cells were flow cell sorted and lysed in RLT buffer of RNeasy Micro Kit (Quiagen) supplemented with β-mercaptoethanol. RNA was extracted according to manufacturer instructions. For each sample, RNA obtained was converted into cDNA using iScript cDNA synthesis kit (Biorad). The expression levels of Helios were then assessed by quantitative PCR, using Quantitec Sybr Green PCR kit (Quiagen). Samples were run for 40 cycles on an ABI 7900HT Fast Real-Time PCR System (Applied Biosystems). The relative quantity of transcripts encoding the gene of interest was determined in each sample by normalization to Gapdh (glyceraldehyde phosphate dehydrogenase) housekeeping gene by using the standard ΔCt method.

14. Statistics

Statistical significance was calculated with the Mann-Whitney test, Kruskal-Wallis test with Dunn's comparison post-test and Two-way Anova tests with Bonferroni comparison post-test (Prism 5, GraphPad Software). Kaplan-Meier and Log-rank Mantel Cox statistical analyses were used to depict mouse survival. P values<0.05; <0.009 and <0.0009 are marked with (*), (**), (***) symbols.

### Introduction

Like adaptive immune responses, innate immune functions must be tightly regulated to be effective without being armful. Natural killer (NK) cells are lymphocytes involved in innate immune responses, which have developed mechanisms to adapt their responses to the host. This adaptation of NK cell reactivity is illustrated by their education mediated via inhibitory receptors which promotes NK cell responsiveness upon interaction with self-MHC class I molecules (1-3, 4, 5-9). NK cells are cytolytic and cytokine-producing lymphocytes which eliminate transformed and microbe-infected cells and participate in the shaping of adaptive immune responses (10, 11).

### Results

1. Identification of a Mouse Pedigree with Hyper-Reactive NK Cells

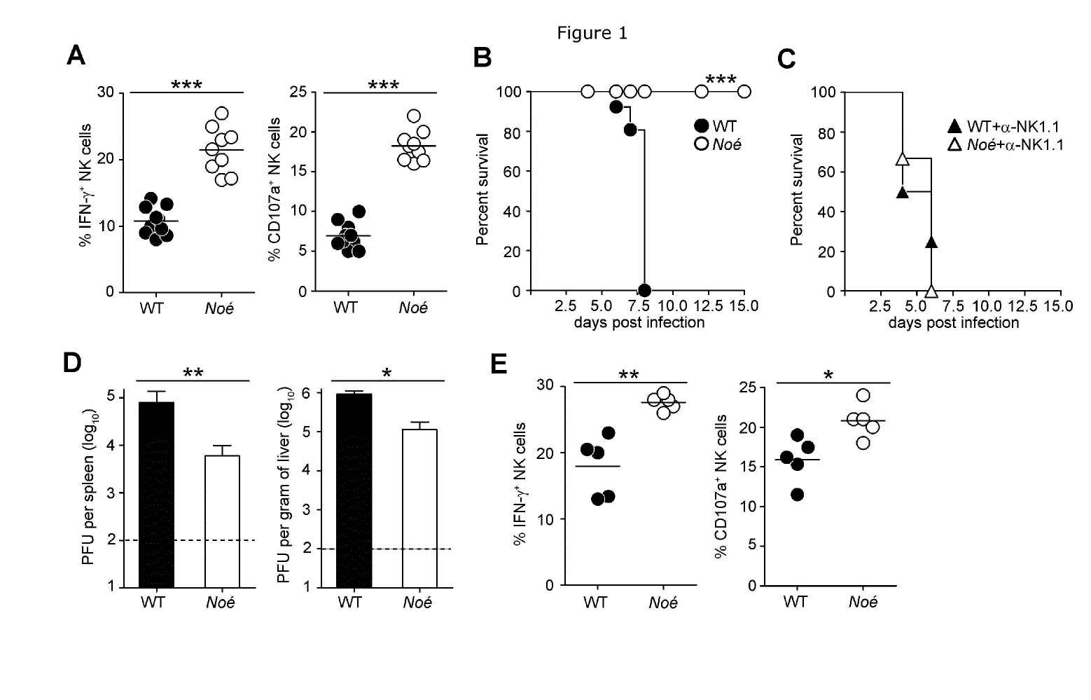

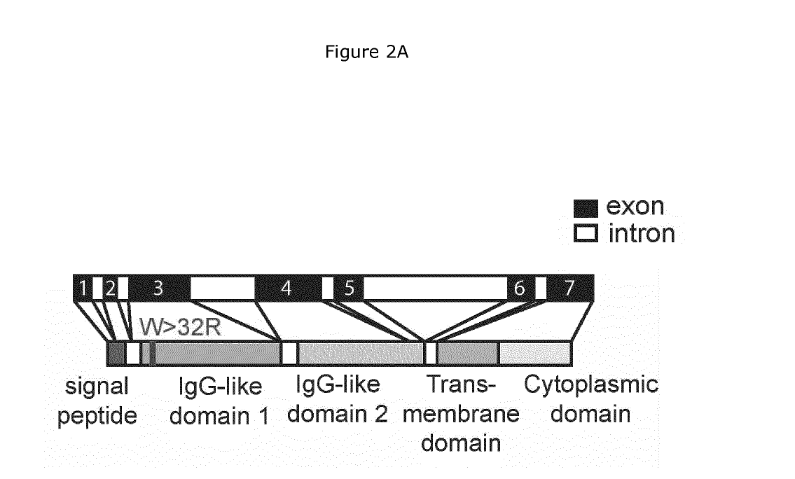

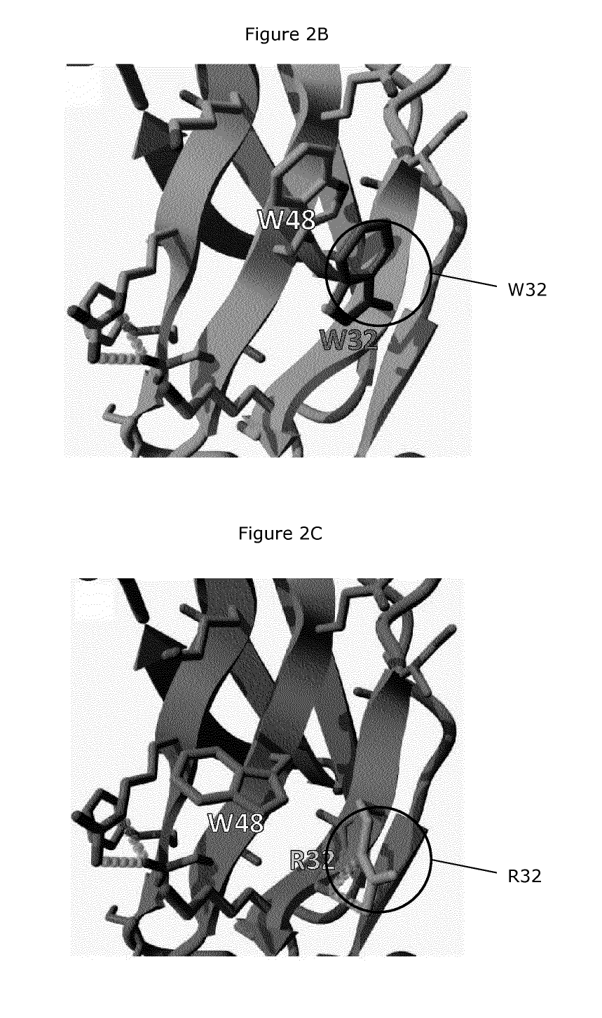

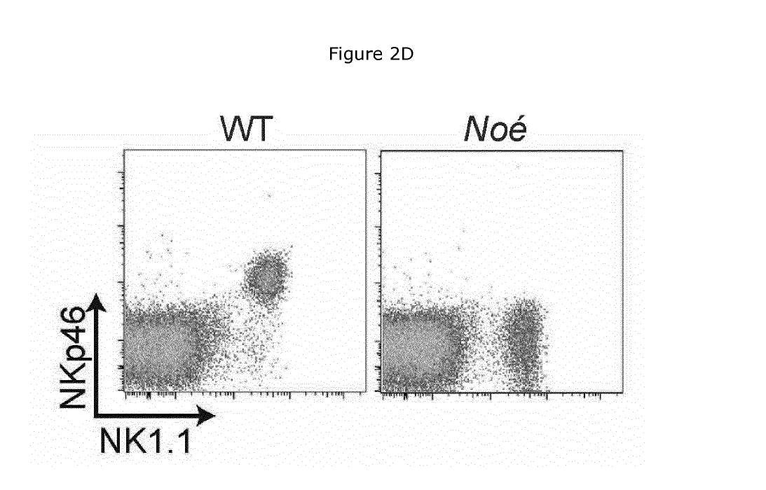

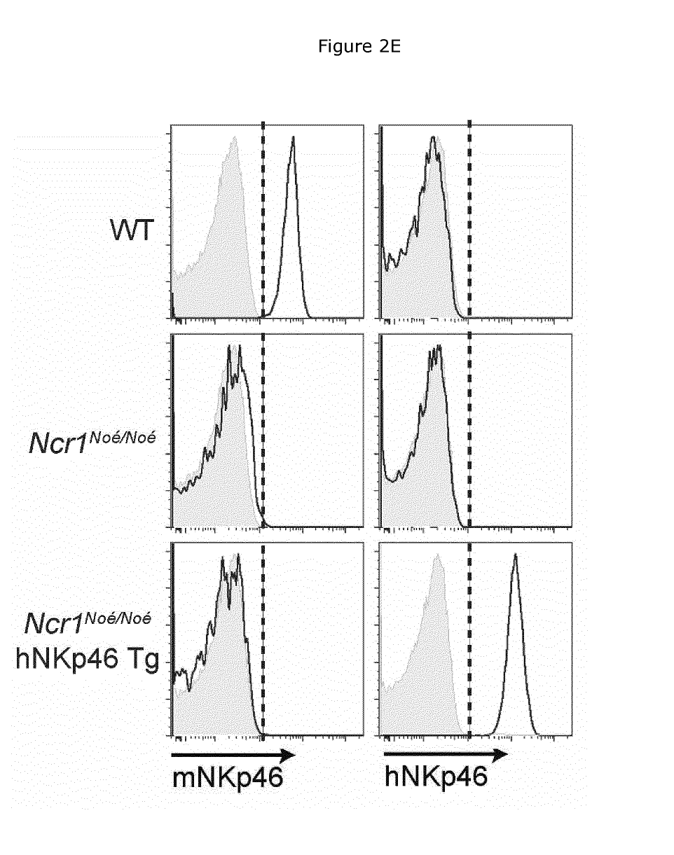

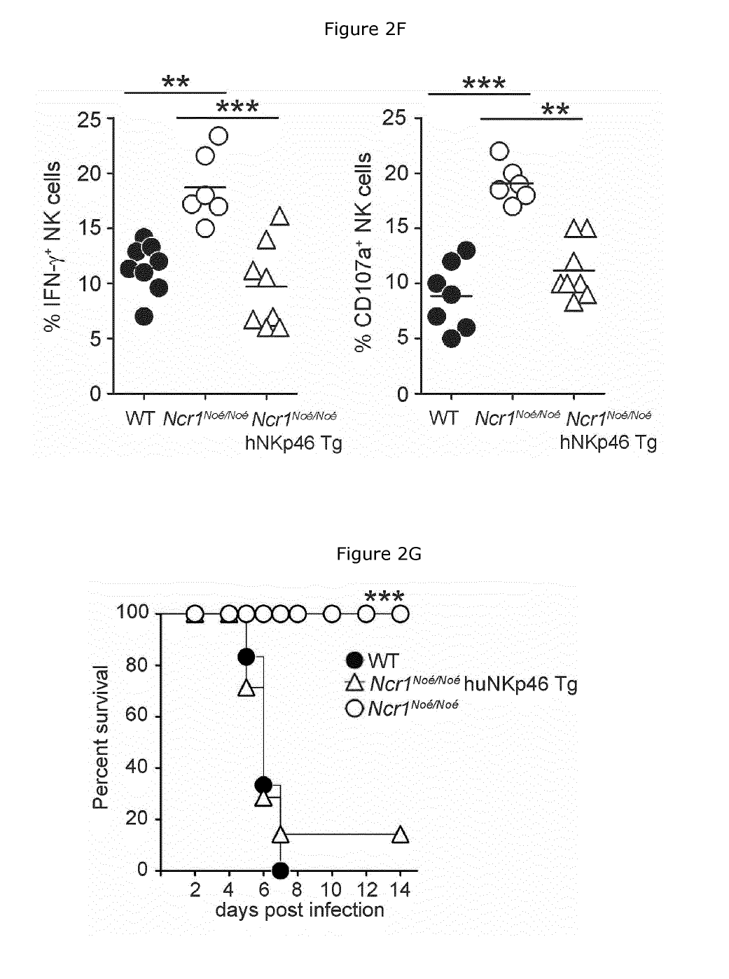

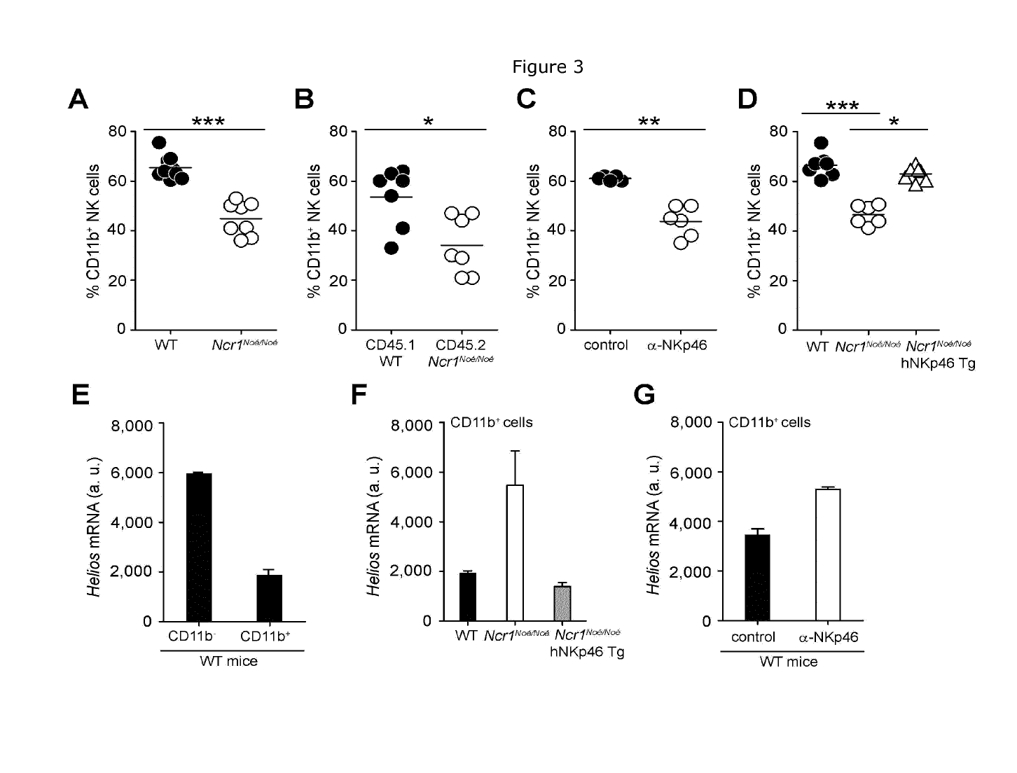

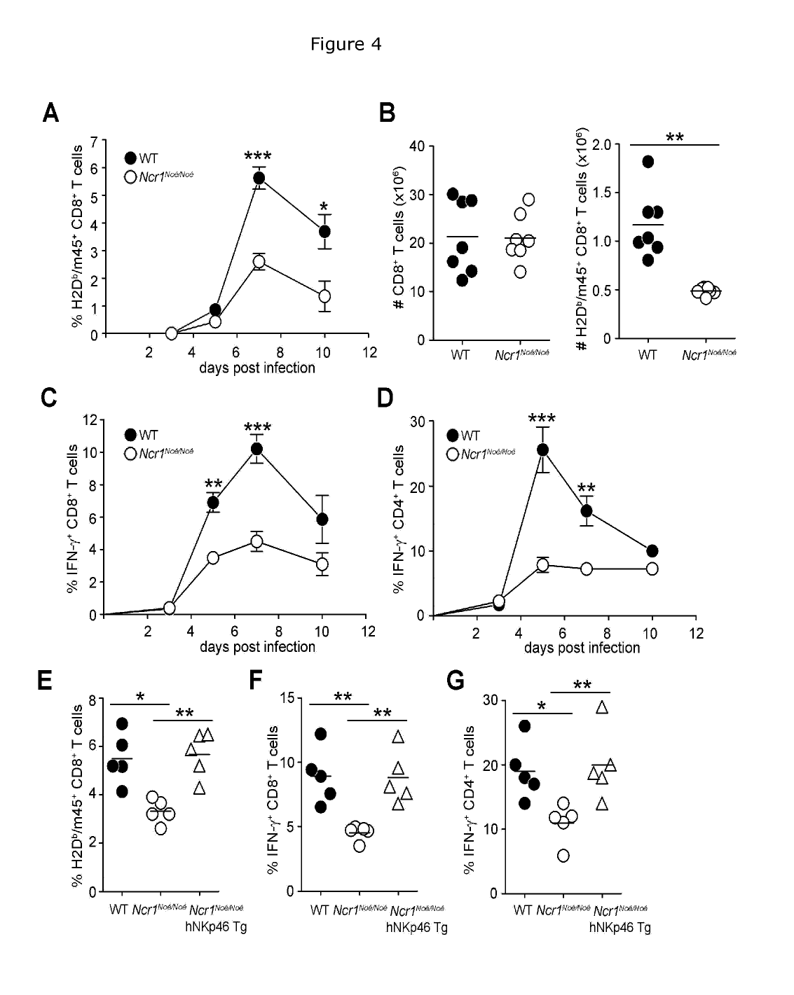

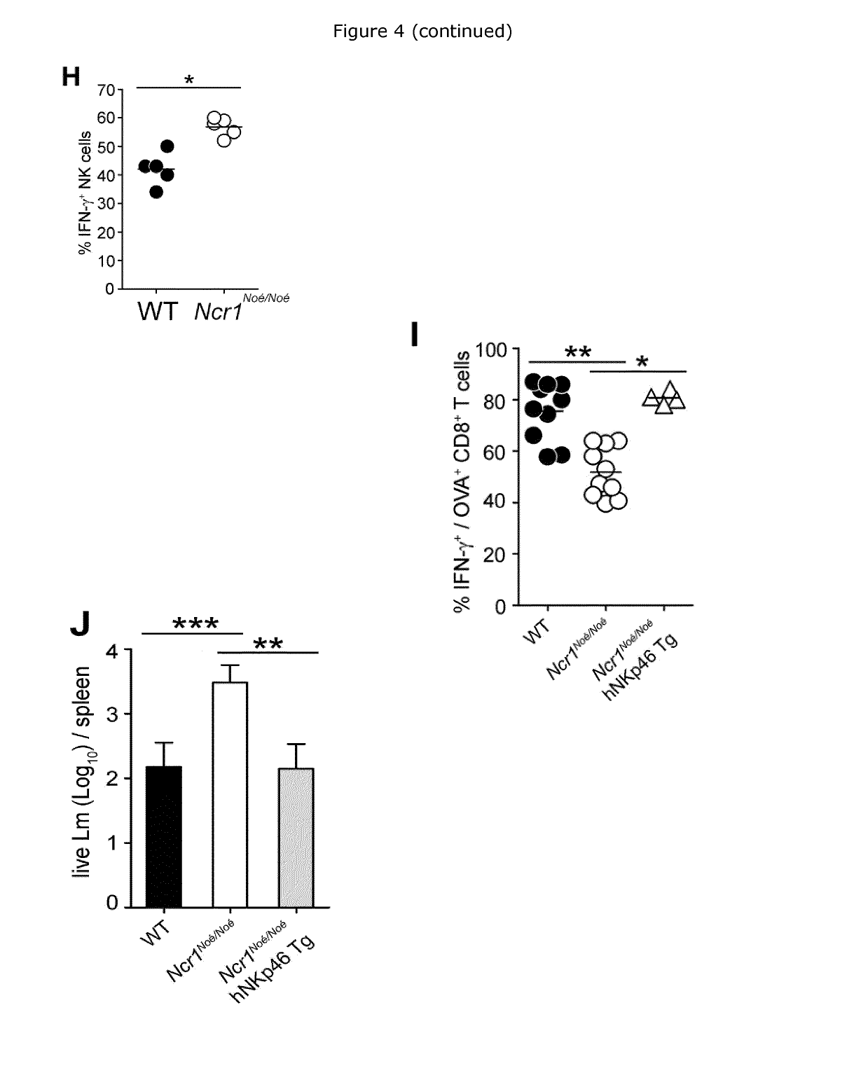

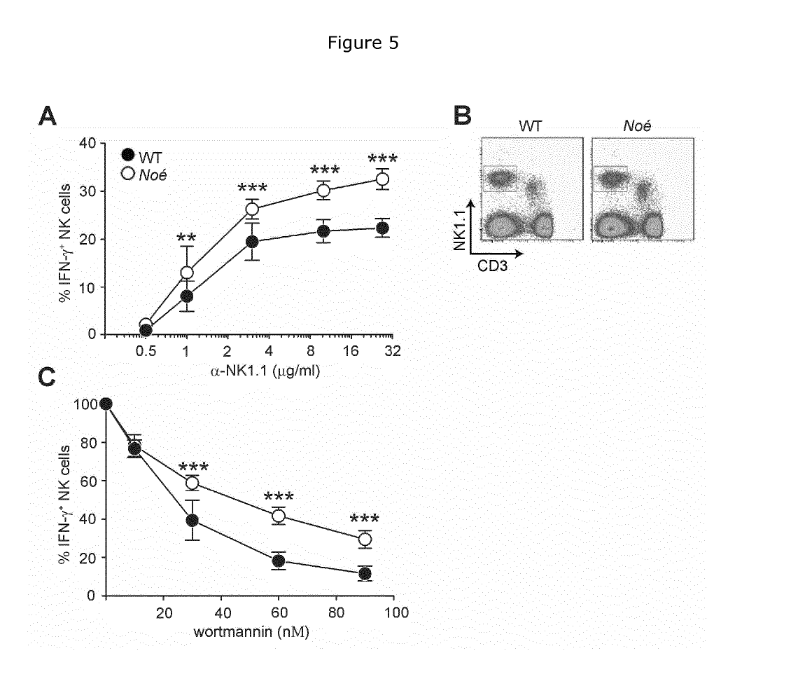

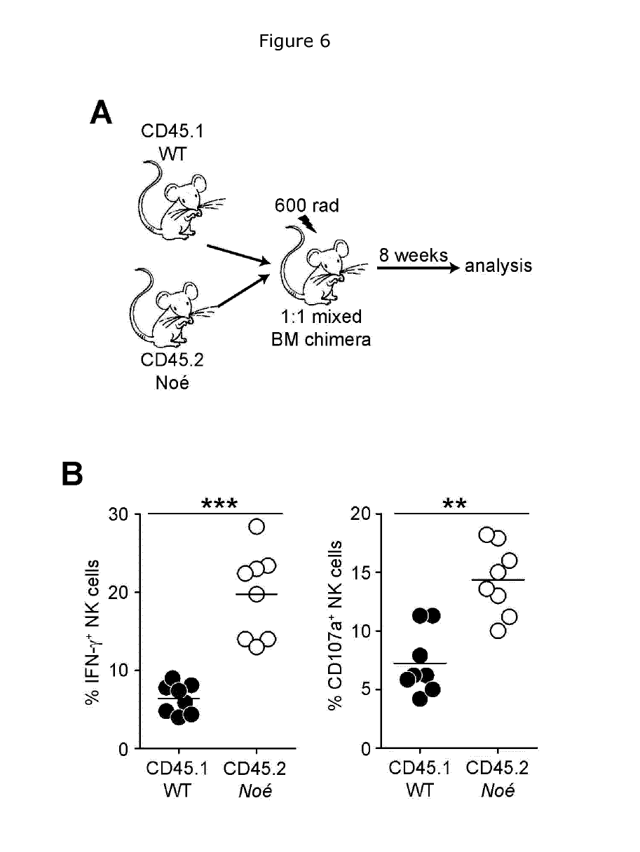

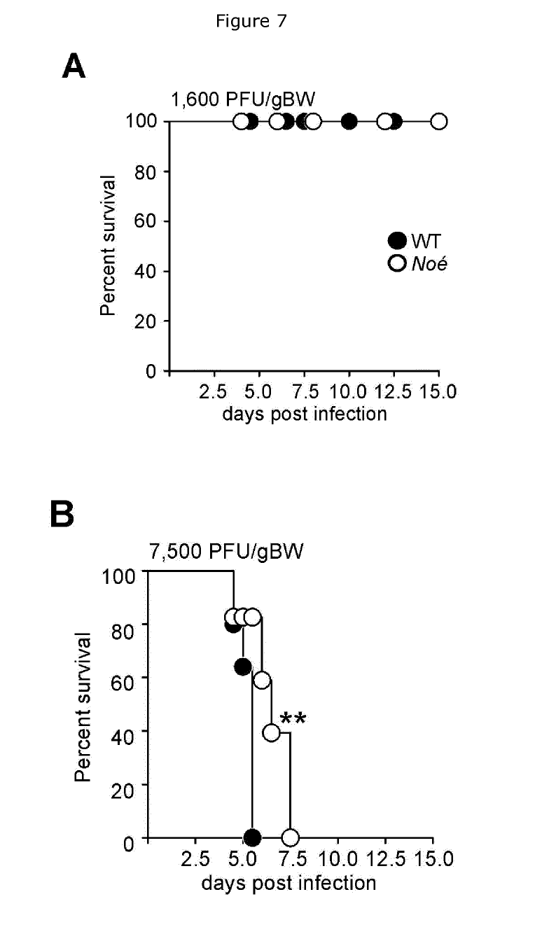

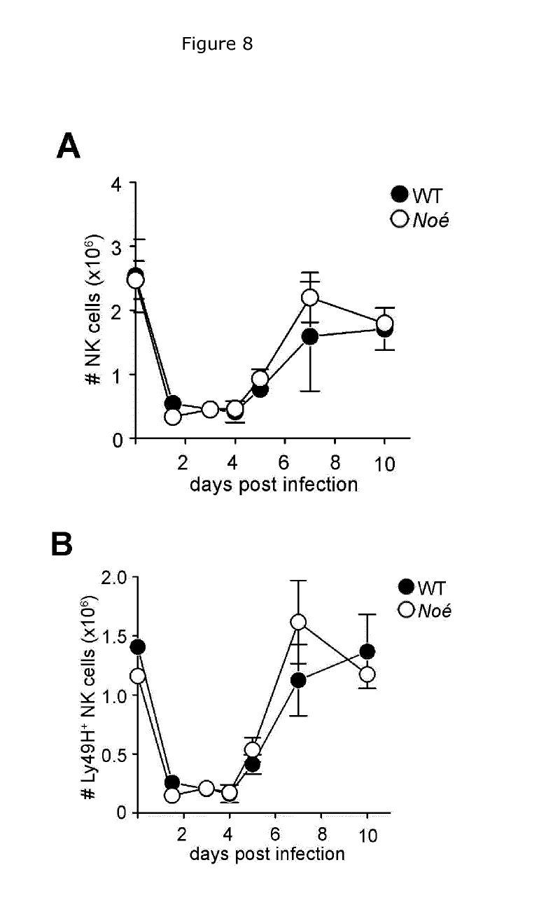

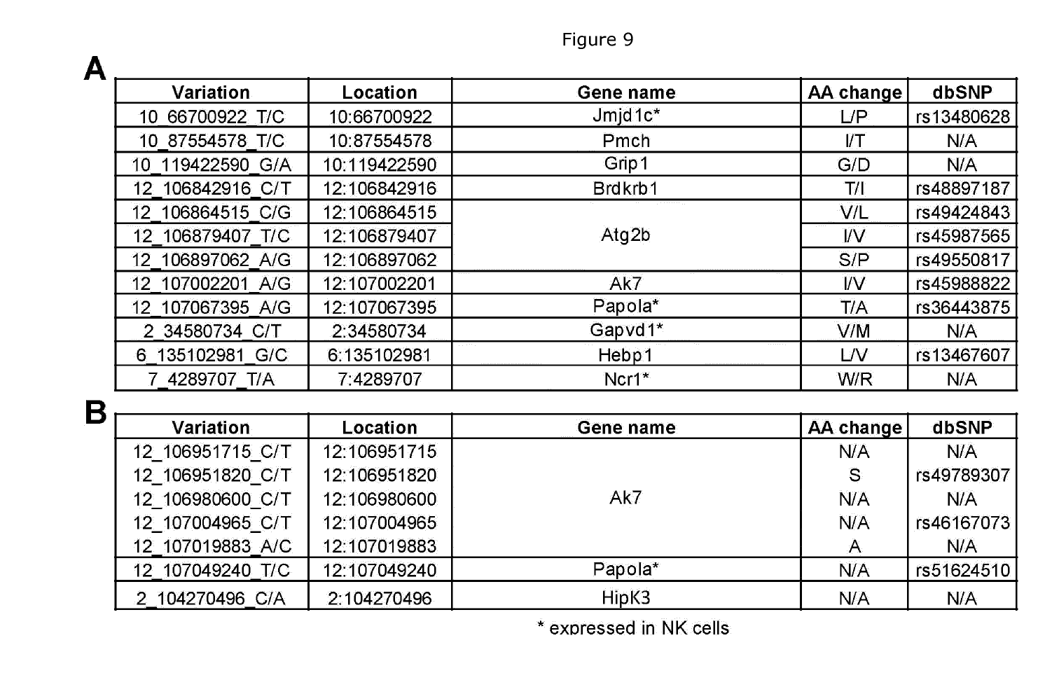

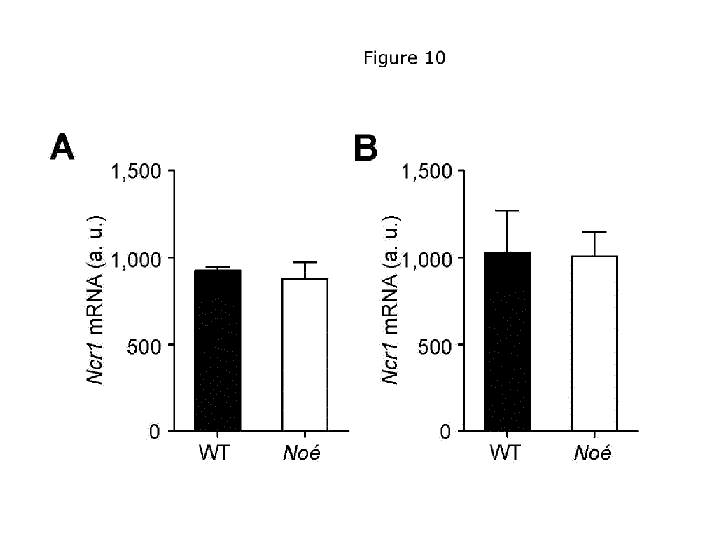

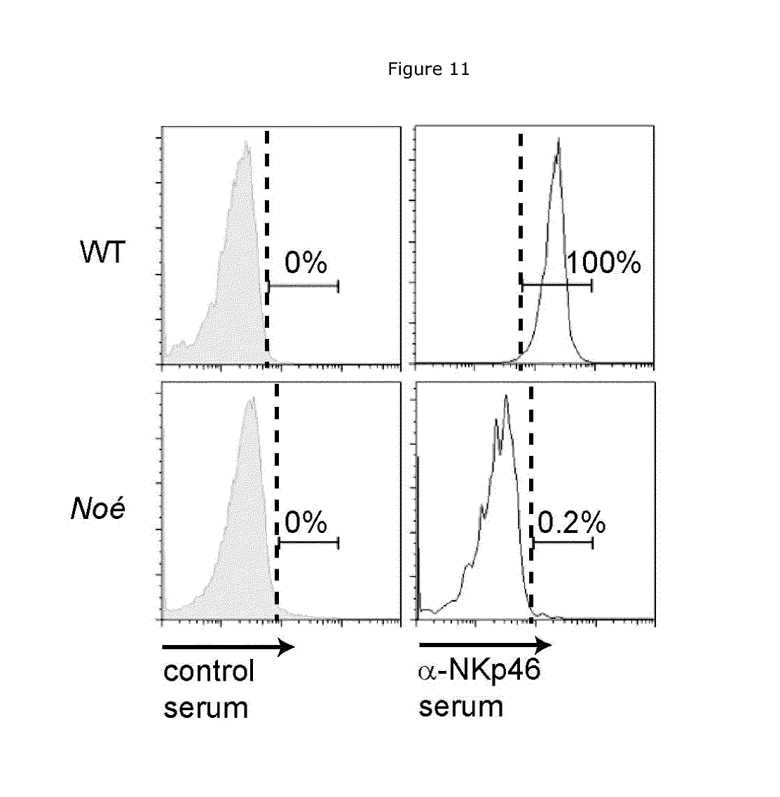

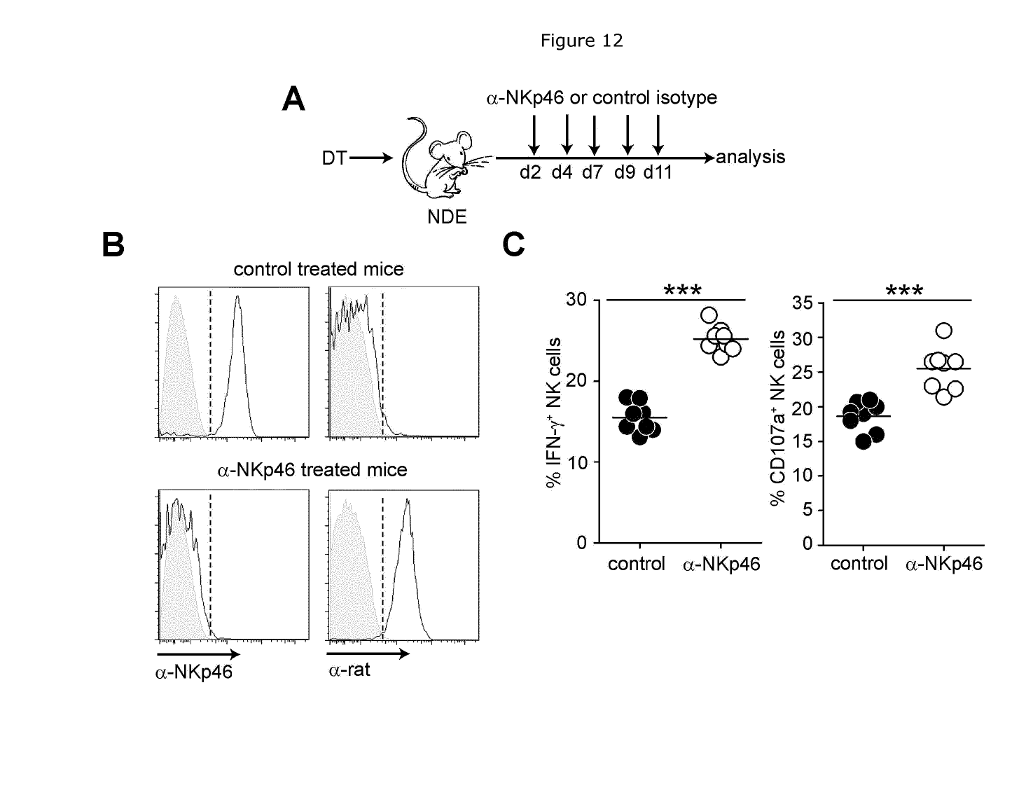

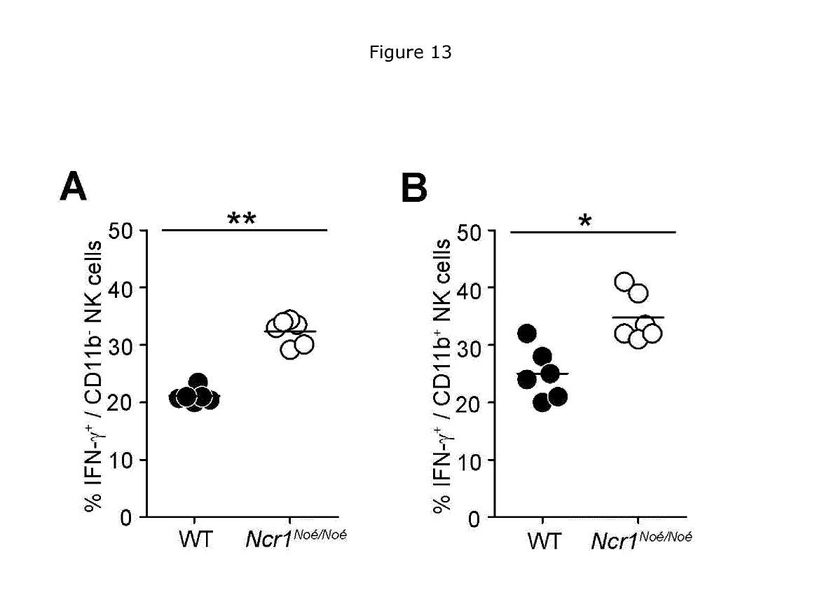

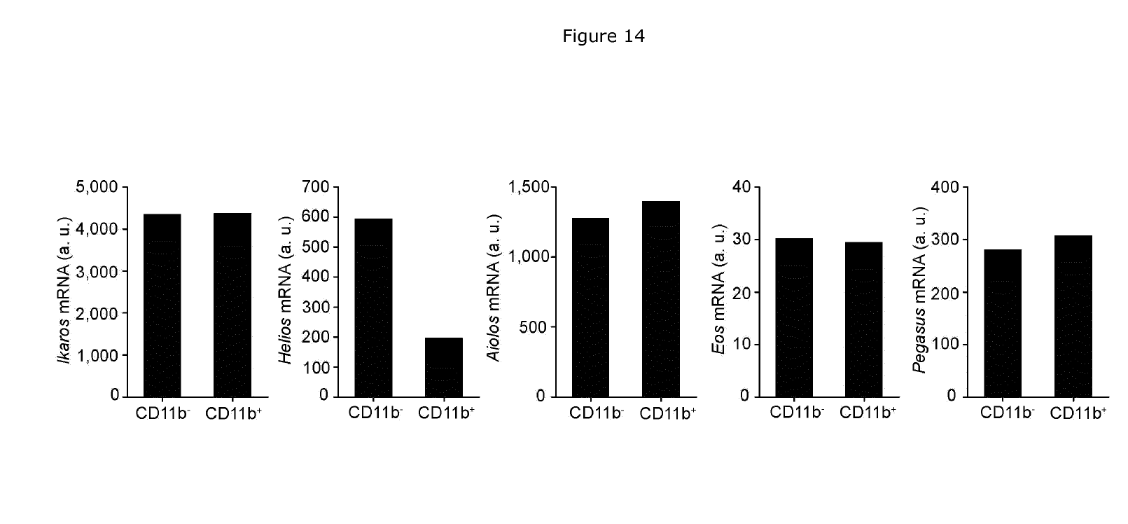

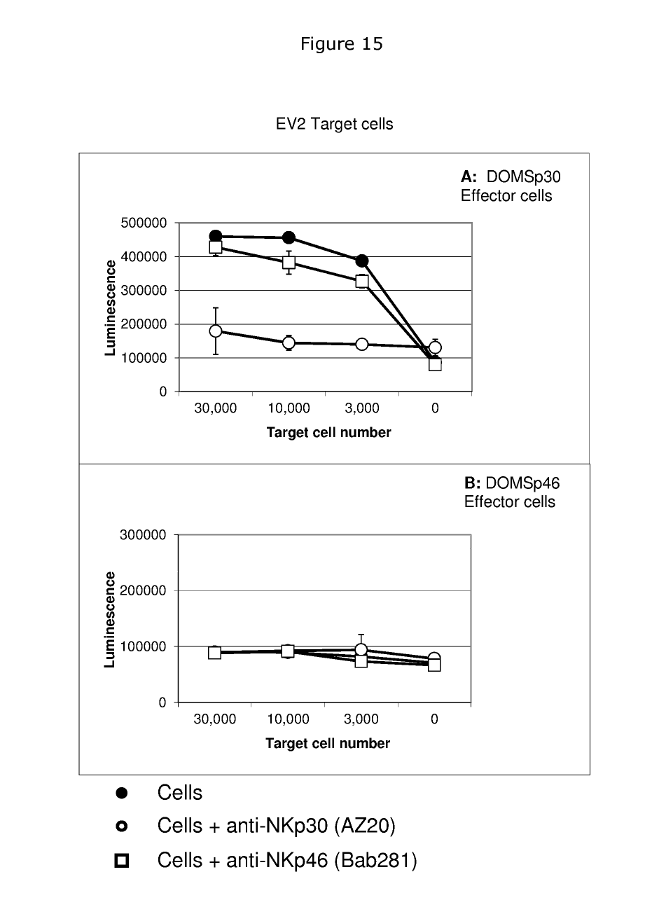

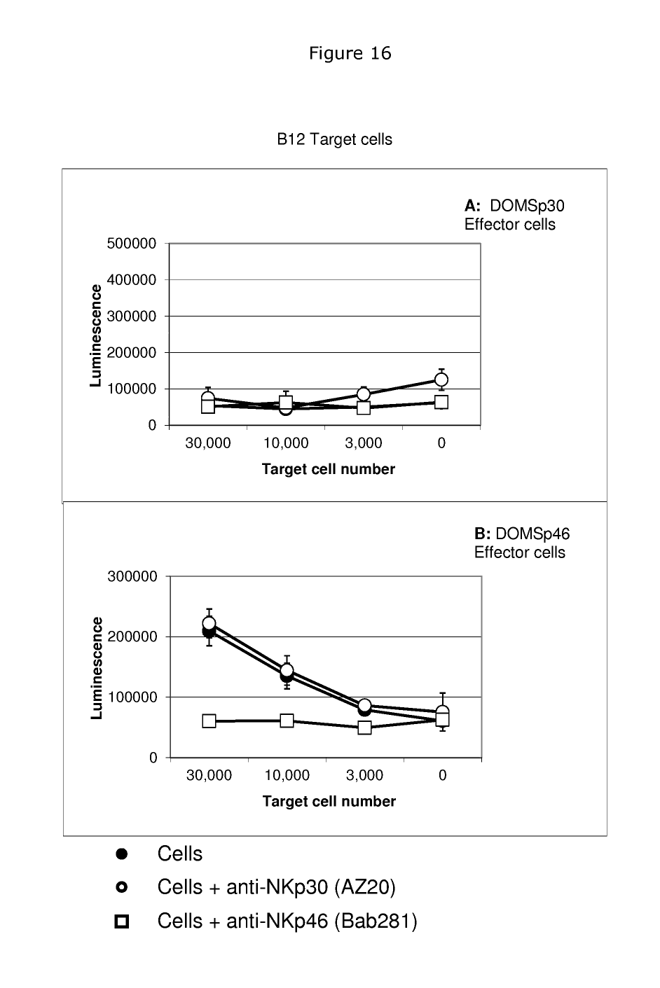

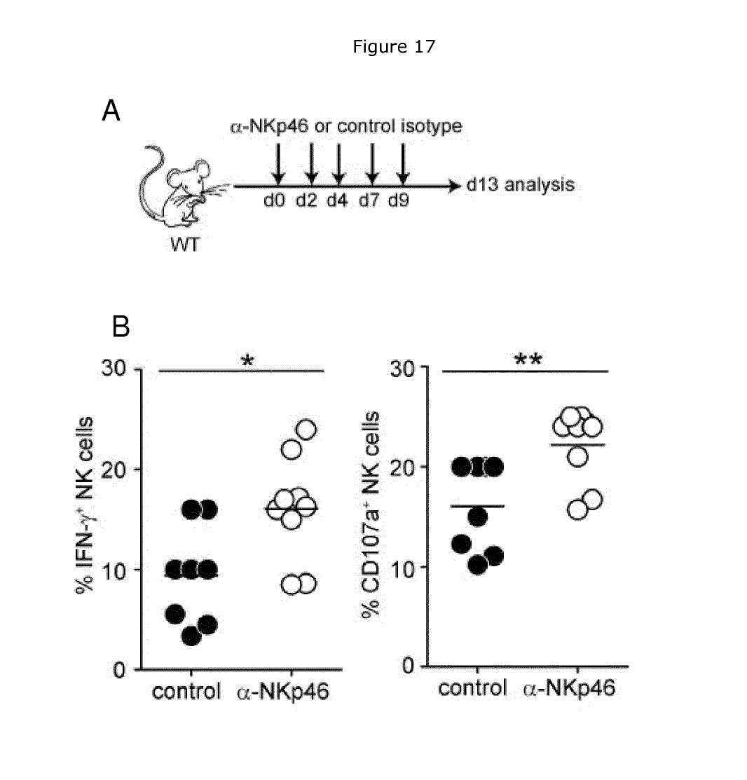

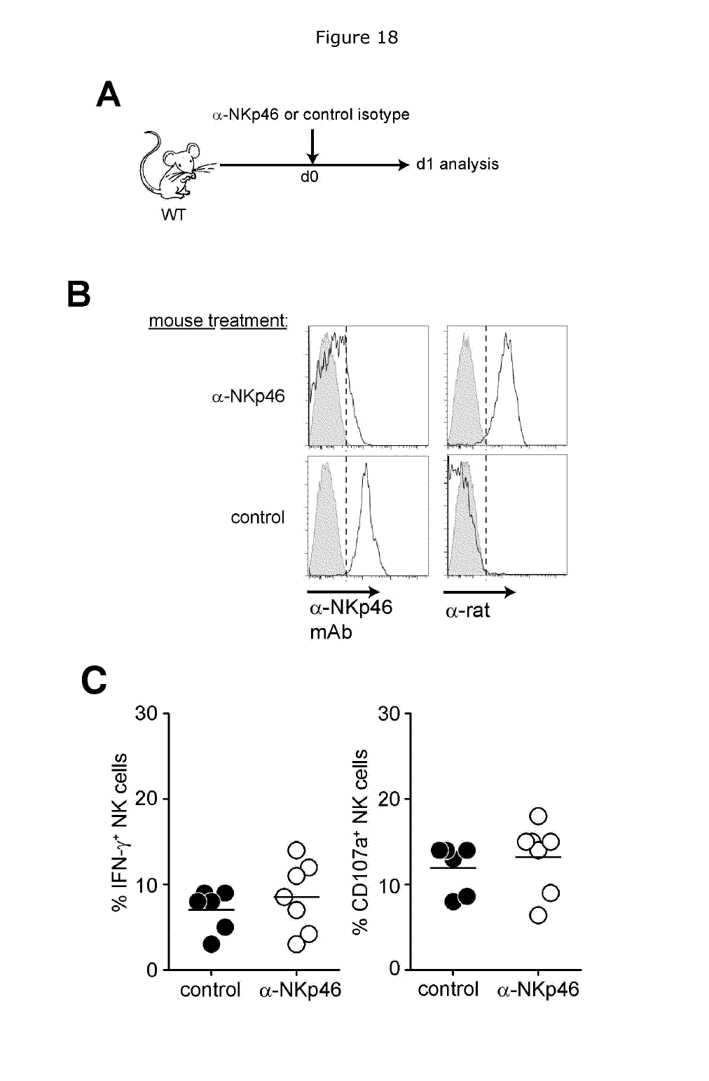

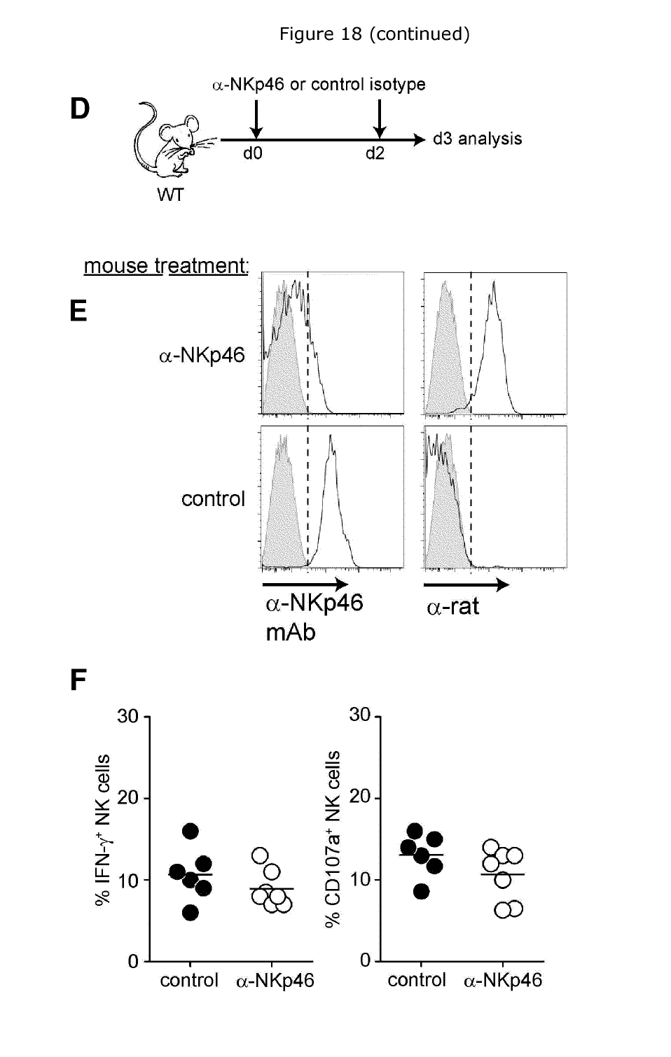

While screening for an altered NK cell phenotype in mice homozygous for ENU-induced mutations, we identified a mouse pedigree with hyper-reactive NK cells in in vitro responses to the prototypical NK cell tumor target, YAC-1. This mouse was bred to establish a homozygous stock on a pure C57BL/6 background. The phenotype was referred as to Noé and appeared to result from a single autosomal recessive mutation. In response to YAC-1 cells, the frequency of IFN-γ-producing cells was increased by 99%±12% (mean±SD, P<0.0001) in IL-2 activated NK cells from Noé mice compared to WT mice (FIG. 1A). In the same test, the frequency of NK cell degranulation detected by CD107a surface expression was increased (162%±12%, P<0.0003) in Noé NK cells (FIG. 1A). Further, resting Noé NK cells were also more responsive when stimulated by monoclonal antibody (mAb) cross-linking of the NK1.1 activating receptor, which is not involved in YAC-1 recognition (FIG. 5A, P<0.0001). This increased reactivity of Noé NK cells was not associated with an increase in NK1.1 cell surface expression (FIG. 5B), and was also observed upon cross-linking of Ly49D and NKG2D surface receptors. In addition, Noé NK cells exhibited a reduced sensitivity to the phosphatidyl-inositol-3 kinase inhibitor wortmannin which prevents signal transduction (FIG. 5C, P<0.0001). Thus Noé NK cells were characterized by a broad increased reactivity in vitro, compatible with a profound change in the threshold of NK cell responses.

To assess whether the Noé NK cell phenotype was cell autonomous, we first reconstituted lethally irradiated recipient mice with a 1:1 mix of bone marrow (BM) cells from WT (CD45.1⁺) and Noé (CD45.2⁺) mice (FIG. 6A). CD45.2⁺ Noé NK cells exhibited a 3-fold (207%±30%, P=0.0002) increase in the percentage of IFNγ-producing cells and a 2-fold increase (98%±19%, P=0.0038) in expression of CD107a⁺, compared to CD45.1⁺ WT NK cells (FIG. 6B). The hyper-responsiveness of Noé NK cells thus relied on an intrinsic cellular modification.

We then tested whether the Noé NK cells were hyper-responsive in vivo by infecting Noé and WT mice with the mouse cytomegalovirus (MCMV). In the C57BL6 background, NK cells play a key role in the early control of MCMV infection via the recognition of the viral protein m157 by the activating receptor Ly49H (12-14). While both Noé and WT animals survived when infected with low dose of MCMV (FIG. 7A), at intermediate doses only Noé mice survived (FIG. 1B, P<0.0001). At high doses that were lethal for both strains, Noé mice survived significantly longer than WT animals (FIG. 7B, P=0.0076). This improved resistance of Noé mice was dependent on NK cells since the injection of a depleting anti-NK1.1 mAb abrogated the ability of Noé mice to survive to an intermediate dose (FIG. 1C). Consistent with their resistance to MCMV infection, Noé mice exhibited a 10 fold reduction (n=10) of MCMV loads in spleen and liver as compared to WT animals 4 days post infection (FIG. 1D). While no change in the counts of the whole NK cell population and Ly49H⁺ NK cells was detected between Noé and WT mice (FIG. 84), the frequencies of IFN-γ-producing and CD107a⁺ NK cells ex vivo were increased by respectively 53%±11% and 33%±8% (n=5) in Noé mice 1.5 days post-infection (FIG. 1E). These results thus supported that Noé mice were more resistant to MCMV infection as a consequence of an improved responsiveness of NK cells in vivo.

2. Identification of NKp46 Mutation

To identify the recessive mutation responsible for the broad hyper-responsiveness of Noé NK cells in vitro and in vivo, we resequenced the whole genome of a Noé and a control WT mouse using the Illumina GA2X sequencer. Within the nucleotides covered by the reads, we identified 16 homozygous in the Noé mouse sequence potentially impacting protein expression or function, 9 non-synonymous mutations in open reading frames and 7 mutations in splice sites (FIG. 9A, B). Among these mutations, only 4 concerned genes expressed in NK cells and thus appeared as candidates for being responsible for the NK cell intrinsic Noé phenotype. We focused our attention on the Ncr1 gene which encodes for the NK cell activating receptor NKp46 that is conserved in all mammalian species tested so far and expressed by all mature NK cells (15). By sequencing the Ncr1 gene and coding cDNA, we confirmed the presence of a single base pair T 1505 in A transversion in the third exon of the Ncr1 gene in Noé mice. This mutation transformed tryptophan 32 into an arginine (W>32R) into the middle of the first 6-sheet of the first extracellular Ig-like domain of NKp46 (FIG. 2A). In the native protein, W32 is predicted to make a π-stacking interaction with the side chain of W48 (FIG. 2B). Mutating W32 into R32 was inferred to abolish the interaction with W48, and is predicted to induce a displacement of W48 side chain by around 7 Å and impacting on the proper folding of the protein (FIG. 2C). As no change in the amount of Ncr1 transcripts were detected by quantitative RT-PCR (qRT-PCR) in sorted NK cells from Noé mice as compared to WT NK cells (FIG. 10), we hypothesized that the W32R mutation would affect the stability or targeting of NKp46. To directly test this possibility, NK cells from Noé and WT mice were stained with both an mAb and a polyclonal anti-serum which recognizes non-overlapping epitopes on NKp46 (15). We could not detect the NKp46 cell surface expression using these reagents suggesting that the W32R mutation indeed abrogated the cell surface expression of NKp46 on NK cells (FIG. 2D and FIG. 11).

3. Genetic Complementation: NKp46 Involvement in Hyper-Responsiveness of Noé NK Cells

We then used genetic complementation to formally assess the direct involvement of the NKp46^{W32R} mutation in the hyper-responsiveness of Noé NK cells. Mouse and human NKp46 proteins are highly homologous and the human NKp46 protein can recognize mouse tumor cells lines (16), suggesting that we could use the human NKp46 protein to complement the Noé phenotype. Mice homozygous for the W32R mutation, also referred to as Ncr1^{Noé/Noé} mice, were crossed with human NKp46 transgenic mice (huNKp46 Tg), in which the expression of the human NKp46 protein is restricted to NK cells (15). We selected mice lacking mouse Nkp46 expression but expressing the human Nkp46 protein (Ncr1^{Noé/Noé} huNKp46 Tg) and compared them to non transgenic Ncr1^{Noé/Noé} littermates (FIG. 2E). Upon co-culture with YAC-1 tumor targets, the reactivity of IL-2-activated Ncr1^{Noé/Noé} cells complemented with the human NKp46 protein was similar to that measured for WT NK cells (FIG. 2F). Human NKp46 was also able to restore the sensitivity of Ncr1^{Noé/Noé} mice to MCMV infection (FIG. 2G). Altogether, these data showed that the NKp46^{W32R} mutation was responsible for the NK cell phenotype in Ncr1^{Noé/Noé} mice.

4. Engagement of NKp46 with an Endogenous Ligand During NK Cell Development Down-Regulates their Responsiveness and Blockade by Anti-NKp46 mAb

NKp46 is associated with ITAM-bearing polypeptides such as CD3ζ and FcRγ, which transduce potent activating signals upon triggering (16). It has been reported that NK cells contribute via NKp46 to type I diabetes through the destruction of pancreatic n-islets, as well as to the control of influenza infection and tumor development (17-20). These data and the reported interaction between NKp46 and viral hemagglutinins (21) have prompted intensive investigations on the biological function of NKp46. Yet, the identification of a cellular ligand for NKp46 is still missing. The phenotype of the Ncr1^{Noé/Noé} mice suggested that NKp46 may be unexpectedly involved in setting the threshold of NK cell responsiveness. To assess whether the engagement of NKp46 by a putative endogenous unknown ligand was required to set the reactivity of NK cells, we sought to neutralize NKp46 during NK cell development in WT mice. We took advantage of the NKDTR/EGFP (NDE) transgenic mouse model in which diphteria toxin treatment depletes NK cells (15). During NK cell repopulation, we treated animals either with the anti-NKp46 mAb or with an isotype control mAb for 2 weeks (FIG. 12A). Flow cytometry analysis of NK cells in anti-NKp46 mAb-treated mice showed that all the sites recognized by the anti-NKp46 mAb were fully occupied and that NKp46 was still expressed at the NK cell surface as the injected anti-NKp46 mAb could be revealed by a cell surface staining with a secondary antibody (FIG. 12B). Upon co-culture with YAC-1 tumor targets, NK cells isolated from anti-NKp46 mAb-treated animals exhibited a 60%±8% and 38%±11% increase in the frequencies of IFN-γ-producing and CD107a⁺ NK cells respectively as compared to control animals (FIG. 12C). Thus, the treatment of WT mice with the anti-NKp46 mAb mimicked the phenotype observed in Ncr1^{Noé/Noé} mice. These data supported the notion that the engagement of NKp46 with an endogenous ligand during NK cell development down-regulates their responsiveness.

NKp46 is expressed early during NK cell differentiation in BM after the induction of NK1.1 expression and before the expression of CD11b. We monitored the frequency of CD11b⁺ NK cells in the bone marrow of Ncr1^{Noé/Noé} mice to test whether the lack of expression of NKp46 could affect NK cell maturation. Ncr1^{Noé/Noé} mice exhibited a 31%±4% (n=9) reduction in the frequency of CD11b⁺ NK cells as compared to WT controls (FIG. 3A). This defect was also observed in mixed BM chimera as well as in NDE mice treated with DT and anti-NKp46 mAb in vivo (FIG. 3B, C). The genetic complementation of the Noé mutation in Ncr1^{Noé/Noé}×huNKp46 Tg mice restored the frequency of mature CD11b⁺ NK cells to that observed in WT animals (FIG. 3D). NKp46 expression was thus necessary to induce proper NK cell maturation. However, the hyper-responsiveness of Ncr1^{Noé/Noé} NK cells was not merely the consequence of an alteration in the transition from CD11b^{low} to CD11b^{high} during NK cell maturation, as both CD11b⁻ and CD11b⁺ NK cells subsets were hyper-responsive to NK1.1 stimulation in Ncr1^{Noé/Noé} mice and less sensitive to wortmannin (FIG. 13). Altogether these data indicated that the lack of NKp46 in Nc1^{Noé/Noé} mice during NK cell development modified their maturation program, engaging them into a pathway characterized by an altered cell surface phenotype and a hyper-reactivity in vitro and in vivo.

5. NKp46 Modulates Helios During NK Cell Maturation

To further dissect the mechanisms by which NKp46 controls NK cell tuning, we focused on transcription factors that are modulated at the transition from CD11b⁻ to CD11b⁺ during the maturation of NK cells in WT mice. Using pan-genomic transcriptomic analysis, we identified that the Ikzf2/Helios gene, which encodes a member of the Ikaros transcription factor family, was differentially regulated in CD11b⁻ as compared to CD11b⁺ WT NK cells (FIG. 14). Previous studies have reported that lymphocytes from mice carrying mutations in Ikaros transcription factor family exhibited defects in maturation, hyper-reactivity upon antigenic stimulation and increased resistance to inhibitors of signal transduction (22). Therefore, Helios was a potential candidate responsible for hyper-responsiveness of Noé NK cells. We confirmed by qRT-PCR that CD11b+NK contained 3 times less Helios mRNA as compared to CD11b⁻ NK cells (FIG. 3E). The reduced expression of the transcription factor Helios was thus associated with NK cell maturation in WT mice. In contrast, Helios transcripts were twice as highly expressed in CD11b⁺ NK cells of Ncr1^{Noé/Noé} as compared to WT mice (FIG. 3F). The genetic complementation of the Ncr1^{Noé} mutation in Ncr1^{Noé/Noé}×huNKp46 Tg mice restored the quantity of Helios transcripts to that detected in WT mice (FIG. 3F). Additionally, the treatment of WT mice with the blocking the anti-NKp46 mAb led to an increase in Helios transcripts (FIG. 3G). Altogether, these data indicated that NKp46 expression and engagement in vivo is necessary for both the down-regulation of the Helios transcription factor in CD11b⁺ mature NK cells and the regulation of NK cell responsiveness. As the ectopic expression of Helios in B cells induced their hyper-responsiveness to antigen (23), our findings supported that Helios plays a pivotal role in the phenotype of Ncr1^{Noé/Noé} mice, and that the engagement of NKp46 during NK cell development set the responsiveness of NK cells via the silencing of Helios.

6. Modulation of T Cell Response by Hyper-Reactive NK Cells

NK cells have been reported to limit the T cell response during MCMV infection either by limiting the amount of antigen or by killing activated T cells (24, 25). Therefore, although hyper-responsiveness of NK cells improved mice survival to MCMV infection, we sought to investigate whether this activity could also impact on the adaptive immune responses. We thus monitored the frequencies of MCMV-specific H2D^{b}/m45⁺ CD8+ T cells over the course of MCMV infection in Ncr1^{Noé/Noé} and WT animals at low infectious dose of MCMV (FIG. 4A). Results showed that Noé mice exhibited a 2- to 3-fold reduction in the frequency of splenic CD8⁺ T cells specific for the immunodominant MCMV peptide m45 presented by H2D^{b} at 7 and 10 days after infection, respectively. We also measured a 2.4-fold reduction in the absolute number H2D^{b}/m45⁺ CD8+ T cells but not in total CD8⁺ T cells in Ncre1^{Noé/Noé} as compared to WT mice (FIG. 4B). In addition, we monitored the frequencies of IFN-γ-producing in CD8⁺ T cells and CD4⁺ T cells after in vitro re-stimulation (FIGS. 4C and D). Ncr1^{Noé/Noé} mice exhibited a 2- to 3-fold reduction respectively in the frequencies of IFN-γ-producing CD8⁺ T cells and CD4⁺ T cells at the peak of the response. In Ncre1^{Noé/Noé}×huNKp46 Tg mice, the level of H2D^{b}/m45⁺ CD8⁺ T cells (FIG. 4E) and IFN-γ-producing cells in CD8⁺ T cells (FIG. 4F) and CD4⁺ T cells (FIG. 4G) were restored to that measured in WT animals 7 days post infection. Thus, the hyper-responsiveness of NK cells improved virus control at high doses of virus but limited subsequent T cell responses.

Hyperresponsive NK cells may therefore be advantageous initially, subsequently becoming disadvantageous during a secondary challenge if the capacity to mount a memory immune response is impaired. We tested this hypothesis, by analyzing the CD8⁺ T-cell protective immunity generated in response to intracellular bacteria Listeria monocytogenes (Lm) expressing ovalbumin (OVA), which is completely cleared after primary infection, unlike MCMV. During Lm infection, NK cells are activated by cytokines, such as IL-12 (Tripp et al (1993) Proc. Natl. Acad. Sci. USA. 90, 3725). Consistent with their broad hyperresponsiveness, the frequencies of IFN-γ-producing NK cells were 36%±3.2% higher in Ncr1^{Noé/Noé} mice than in the WT 24 hours after infection with Lm-OVA (FIG. 3H; P=0.0119). Following primary Lm-OVA infection and rechallenge 30 days later, the percentages of memory Lm-OVA-specific CD8⁺ T cells capable to produce IFN-γ were 35%±4.4% and 36%±4% lower in Ncr1^{Noé/Noé} mice than in WT mice and Ncre1^{Noé/Noé} NKp46 Tg mice, respectively (FIG. 3I; P<0.01 and P<0.05, respectively). This alteration in the quality of Lm-OVA-specific CD8⁺ memory T cells in Ncr1^{Noé/Noé} mice was associated with a bacterial load in the spleen 12 times higher than that in WT mice and Ncre1^{Noé/Noé}huNKp46 Tg littermates (FIG. 3J; P<0.001 and P<0.01, respectively). Thus, the hyperresponsiveness of NK cells during the T cell-priming phase affected the generation of fully protective memory T cells. Given the extremely high level of conservation observed for NKp46 in mammalian species, these results suggest that the regulation of NK cell innate immune responses by NKp46 henceforth referred as to “disarming”, was selected during evolution to favor adaptive immune responses. Evolution in mammals may have resulted in the counterselection of hyperreactive innate immunity mechanisms, despite their efficiency during the first encounter with a pathogen to favor the emergence of adaptive immune responses, which efficiently control high doses of pathogens upon re-exposure. NKp46 blockade may therefore be harnessed, to enhance NK cell effector functions, as a novel immunotherapeutic strategy to limit inappropriate or harmful T cell responses.

### CONCLUSION

So far, the dissection of NK cell education has been focused so far on the role of inhibitory receptors for MHC class I. It was shown that the engagement of inhibitory receptors contribute to turn ‘on’ NK cells, a process referred as to arming or licensing (1-3, 26, 27, 28, 29). Our analysis of Ncr1^{Noé/Noé} mice implicated the activating receptors NKp46 as a checkpoint of NK cell tuning and revealed another aspect of NK cell education in which the engagement of NKp46 negatively regulates NK cell responsiveness. These results are consistent with observations performed for other activating receptors in NKG2D-deficient mice (30), or in transgenic mice engineered to express ligands for NKG2D or Ly49H (31, 32). Our results thus support a model as which the engagement of NKp46 with endogenous ligands during NK cell maturation induces the down-modulation of the Helios transcription factor to set the threshold of NK cell responses. Altogether, these data revealed an unappreciated role for the activating receptor NKp46 in the calibration of NK cell responsiveness to the host environment.

### Example 2

### Identification of Blocking NKp46 Antibodies

Anti-NKp46 antibodies were tested for their ability to inhibit NKp46 (inhibit NKp46 activity, signaling and/or ligand binding) using a reporter systems to evaluate whether candidate antibodies block NKp46-ligand induced NK cell lysis of a target cell. The reporter system used the IL-2-producing DO11.10 mouse T cell hybridoma expressing a chimeric receptor formed by either NKp46 or NKp30 extracellular domain fused to CD3ζ (DOMSP46 and DOMSP30 cells, respectively) as described in Schleinitz et al., (2008) Arthritis Rheum. 58: 3216-3223).

### Materials and Methods

DOMSP30 and DOMSP46 reporter cell lines were generated by transduction of the DO.11.10 T cell hybridoma with retroviral particles encoding a chimeric protein in which the intracytoplasmic domain of mouse CD3ζ was fused either to the extracellular portion of NKp30 (DOMSP30) or NKp46 (DOMSP46). Engagement of these chimeric proteins at the cell surface triggers IL-2 secretion. DOMSP30, DOMSP46, or DO.11.10 (20,000 cells/well in 96-well plates) were incubated on anti-NKp30 or anti-NKp46 mAb-coated plates or with Hela EV2 or B12 cell lines (0; 3,000; 10,000 or 30,000 cells/well in 96-well plates). After 20 h, cell supernatants were assayed for the presence of mouse IL-2 in a standard CTLL-2 survival assay using Cell Titer-Glo Luminescent Cell Viability Assay (Promega).

### Results

DOMSP30 reporter cells were activated (as expressed by IL-2 induced CTLL2 cell proliferation) when brought into contact with Hela EV2 cells (FIG. 15A) but not B12 cells (FIG. 16A), showing that Hela EV2 but not B12 cells express a NKp30 ligand. Addition of anti-NKp30 antibodies (clone AZ20) reduced CTLL-2 cell proliferation indicating that the antibodies blocked NKp30 while antibodies to NKp46 (Bab281) did not reduce CTLL-2 cell proliferation (FIG. 15A).

DOMSP46 reporter cells were activated (as expressed by IL-2 induced CTLL2 cell proliferation) when brought into contact with B12 cells (FIG. 16B) but not Hela EV2 cells (FIG. 15B), showing that B12 but not Hela EV2 cells express a NKp46 ligand. Addition of anti-NKp46 antibodies (clone Bab281) reduced CTLL-2 cell proliferation indicating that the antibodies blocked NKp46 while antibodies to NKp30 (AZ20) did not reduce CTLL-2 cell proliferation (FIG. 16B). Bab281 therefore inhibits ligand-induced NKp46 signaling.

### Example 3

### Extended Receptor Saturation is Required In Vivo with Blocking NKp46 Antibodies

Our findings suggested that NKp46 blockade could be harnessed, to enhance NK cell effector functions, as a novel immunotherapeutic strategy. We thus evaluated whether we could modify the responsiveness of NK cells by injecting anti-NKp46 mAb, at steady state, in WT mice (FIG. 17A). Short treatments lasting 24 to 72 hours, were sufficient to saturate NKp46 receptors (FIGS. 18A, B, D and E), but did not increase the reactivity of NK cells in response to YAC-1 tumor targets (FIGS. 18C and F). By contrast, in vivo blockade of NKp46 by the mAb for 13 days was sufficient to enhance NK cell responsiveness (FIG. 17B; *P=0.03 and **P=0.0081). Our results reveal the role of the conserved activating NK cell receptor NKp46 in NK cell function. They also pave the way for the counterintuitive use of blocking anti-NKp46 mAbs, to boost NK cell activity, as a novel immunostimulation strategy for the treatment of patients with cancers or infections, and of particular relevance for patients with T-cell deficiencies, such as those occurring after hematopoietic stem cell transplantation.

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents, The description herein of any aspect or embodiment of the invention using terms such as reference to an element or elements is intended to provide support for a similar aspect or embodiment of the invention that “consists of’,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### REFERENCES CITED IN EXAMPLES

1. E. Vivier et al., Innate or adaptive immunity? The example of natural killer cells. Science 331, 44 (Jan. 7, 2011).
2. D. H. Raulet, Interplay of natural killer cells and their receptors with the adaptive immune response. Nat Immunol 5, 996 (October, 2004).
3. S. Kim et al., Licensing of natural killer cells by host major histocompatibility complex class I molecules. Nature 436, 709 (Aug. 4, 2005).
4. N. C. Fernandez et al., A subset of natural killer cells achieves self-tolerance without expressing inhibitory receptors specific for self-MHC molecules. Blood 105, 4416 (Jun. 1, 2005).
5. N. Anfossi et al., Human NK cell education by inhibitory receptors for MHC class I. Immunity 25, 331 (August, 2006).
6. S. Cooley et al., A subpopulation of human peripheral blood NK cells that lacks inhibitory receptors for self-MHC is developmentally immature. Blood 110, 578 (Jul. 15, 2007).
7. A. Chalifour et al., A Role for cis Interaction between the Inhibitory Ly49A receptor and MHC class I for natural killer cell education. Immunity 30, 337 (March, 2009).
8. N. T. Joncker, N. Shifrin, F. Delebecque, D. H. Raulet, Mature natural killer cells reset their responsiveness when exposed to an altered MHC environment. J Exp Med 207, 2065 (Sep. 6, 2010).
9. J. M. Elliott, J. A. Wahle, W. M. Yokoyama, MHC class I-deficient natural killer cells acquire a licensed phenotype after transfer into an MHC class I-sufficient environment. J Exp Med 207, 2073 (Sep. 6, 2010).
10. P. Brodin, T. Lakshmikanth, S. Johansson, K. Karre, P. Hoglund, The strength of inhibitory input during education quantitatively tunes the functional responsiveness of individual natural killer cells. Blood 113, 2434 (Mar. 12, 2009).
11. S. Guia et al., Confinement of activating receptors at the plasma membrane controls natural killer cell tolerance. Sci Signal 4, ra21 (2011).
12. A. O. Dokun et al., Specific and nonspecific NK cell activation during virus infection. Nat Immunol 2, 951 (2001).
13. H. Arase, E. S. Mocarski, A. E. Campbell, A. B. Hill, L. L. Lanier, Direct Recognition of Cytomegalovirus by Activating and Inhibitory NK Cell Receptors. Science 296, 1323 (2002).
14. H. R. Smith et al., Recognition of a virus-encoded ligand by a natural killer cell activation receptor. Proc. Natl. Acad. Sci. USA 99, 8826 (2002).
15. T. Walzer et al., Identification, activation, and selective in vivo ablation of mouse NK cells via NKp46. Proc Natl Acad Sci USA 104, 3384 (Feb. 27, 2007).
16. A. Pessino et al., Molecular cloning of NKp46: a novel member of the immunoglobulin superfamily involved in triggering of natural cytotoxicity. The Journal of experimental medicine 188, 953 (1998).
17. G. G. Halfteck et al., Enhanced in vivo growth of lymphoma tumors in the absence of the NK-activating receptor NKp46/NCR1. J Immunol 182, 2221 (Feb. 15, 2009).
18. E. Cagnano et al., Expression of ligands to NKp46 in benign and malignant melanocytes. J Invest Dermatol 128, 972 (April, 2008).
19. R. Gazit et al., Lethal influenza infection in the absence of the natural killer cell receptor gene Ncr1. Nat Immunol, (Mar. 26, 2006).
20. C. Gur et al., The activating receptor NKp46 is essential for the development of type 1 diabetes. Nat Immunol 11, 121 (February, 2010).
21. O. Mandelboim et al., Recognition of haemagglutinins on virus-infected cells by NKp46 activates lysis by human NK cells. Nature 409, 1055 (2001).
22. K. Georgopoulos, S. Winandy, N. Avitahl, The role of the Ikaros gene in lymphocyte development and homeostasis. Annu Rev Immunol 15, 155 (1997).
23. S. Dovat et al., Transgenic expression of Helios in B lineage cells alters B cell properties and promotes lymphomagenesis. J Immunol 175, 3508 (Sep. 15, 2005).
24. D. M. Andrews et al., Innate immunity defines the capacity of antiviral T cells to limit persistent infection. J Exp Med 207, 1333 (Jun. 7, 2010).
25. K. Soderquest et al., Cutting edge: CD8+ T cell priming in the absence of NK cells leads to enhanced memory responses. J Immunol 186, 3304 (Mar. 15, 2011).
26. D. H. Raulet, R. E. Vance, Self-tolerance of natural killer cells. Nat Rev Immunol 6, 520 (July, 2006).
27. W. M. Yokoyama, S. Kim, How Do Natural Killer Cells Find Self to Achieve Tolerance? Immunity 24, 249 (March, 2006).
28. P. Brodin et al., Natural Killer cell tolerance persists despite significant reduction of self MHC class I on normal target cells in mice. PloS One 5 (2010)
29. M. T. Orr, L. L. Lanier, Natural killer cell education and tolerance. Cell 142, 847 (Sep. 17, 2010).
30. B. Zafirova et al., Altered NK cell development and enhanced NK cell-mediated resistance to mouse cytomegalovirus in NKG2D-deficient mice. Immunity 31, 270 (Aug. 21, 2009).
31. M. Champsaur, L. L. Lanier, Effect of NKG2D ligand expression on host immune responses. Immunol Rev 235, 267 (May, 2010).
32. J. C. Sun, L. L. Lanier, Tolerance of NK cells encountering their viral ligand during development. J Exp Med 205, 1819 (Aug. 4, 2008).
33. K. Hoebe et al., Identification of Lps2 as a key transducer of MyD88-independent TIR signalling. Nature 424, 743 (Aug. 14, 2003).
34. E. Krieger et al., Improving physical realism, stereochemistry, and side-chain accuracy in homology modeling: Four approaches that performed well in CASP8. Proteins 77 Suppl 9, 114 (2009).
35. J. Schymkowitz et al., The FoldX web server: an online force field. Nucleic Acids Res 33, W382 (Jul. 1, 2005).

1-42. (canceled)

43. A method of treating an individual having a cancer or infectious disease administering to an individual a therapeutically active amount of an antibody that inhibits NKp46 and does not substantially lead to the depletion of NKp46-expressing NK cells.

44. The method of claim 43, wherein said antibody is administered to a mammal for a sufficient period of time to inhibit NKp46 in developing NK cell and cause an increase in the frequency of activated, reactive, cytotoxic and/or IFNγ-producing NK cells in a mammal.

45. The method of claim 43, wherein the antibody inhibits, in a reporter assay, the proliferation of a T cell hybridoma made to express a chimeric protein in which the intracytoplasmic domain of mouse CD3ζ is fused to the extracellular portion of NKp46, when such T cell hybridoma is brought into contact with a NKp46-ligand expressing cell.

46. The method of claim 43, wherein said therapeutically active amount is an amount of such compound that results in substantial saturation of the NKp46 on NK cells for a period of at least about 1 week following administration of the compound.

47. The method of claim 43, wherein said therapeutically active amount is an amount of such compound that results in substantial saturation of the NKp46 on NK cells for a period of at least about 2 weeks following administration of the compound.

48. The method of claim 43, wherein said therapeutically active amount is an amount of such compound that results in substantial saturation of the NKp46 on NK cells for a period of at least about one month following administration of the compound.

49. The method of claim 43, wherein said antibody comprises a heavy chain constant region of the human IgG4 isotype.

50. The method of claim 43, wherein said antibody comprises a human IgG4 heavy chain comprising a serine to proline mutation in residue 241, corresponding to position 228 according to the EU-index.

51. The method of claim 43, wherein said antibody inhibits an interaction between the NKp46 polypeptide and a natural ligand of the NKp46 polypeptide.

52. The method of claim 43, wherein said antibody inhibits NKp46-mediated silencing of Helios transcription factor.

53. The method of claim 43, wherein said antibody competes for binding to a NKp46 polypeptide with an antibody selected from the group consisting of Bab281, 9E2 or 195314.

54. The method of claim 43, wherein the antibody comprises an Fc region that does not substantially bind a human FcγRIII polypeptide (CD16).

55. The method of claim 43, wherein said antibody is an antibody fragment selected from Fab, Fab′, Fab′-SH, F (ab′)2, Fv, diabodies, single-chain antibody fragment, or a multispecific antibody comprising multiple different antibody fragments.

56. A method of producing an antibody for the treatment of a cancer or infectious disease in a mammalian subject, said method comprising the steps of: a) immunizing a non-human mammal with an immunogen comprising a human NKp46 polypeptide or providing a library of antibodies; and b) selecting an antibody from said immunized animal or library that binds to the NKp46 polypeptide with specificity and that inhibits NKp46 signaling.

57. The method of claim 56, wherein said antibody is selected to inhibit an interaction between the NKp46 polypeptide and a natural ligand of the NKp46 polypeptide.

58. The method of claim 56, wherein said antibody is selected to inhibit NKp46-mediated silencing of Helios transcription factor.

59. The method of claim 56, wherein said antibody is selected to increase the frequency of activated, reactive, cytotoxic and/or IFNγ-producing NK cells in a mammal when administered to a mammal for a sufficient period of time to inhibit NKp46 in developing NK cells.

60. The method of claim 56, wherein said antibody competes for binding to a NKp46 polypeptide, with an antibody selected from the group consisting of Bab281, 9E2 and 195314.


SEQUENCE LISTING (3 sequences)

SEQ ID NO: 1 - protein, 304 aa, homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 2 - protein, 526 aa, homo sapiens [sequence omitted]

SEQ ID NO: 3 - protein, 326 aa, Homo sapiens
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK